(19) United States
(12) Patent Application Publication
(10) Pub. No.: US 2010/0291236 A1
(43) Pub. Date: Nov. 18, 2010

(54) OSMIUM COMPOUNDS

(75) Inventors: Peter John Sadler, Penicuik (GB); Anna Frances Acushla Peacock, Leiden (NL); Sabine Helena Van Rijt, Edinburgh (GB); Abraha Habtemariam, Edinburgh (GB)

Correspondence Address: HOXIE & ASSOCIATES LLC, 75 MAIN STREET , SUITE 301, MILLBURN, NJ, 07041, US

(73) Assignee: THE UNIVERSITY OF WARWICK, COVENTRY (GB)

(21) Appl. No.: 12/377,051

(22) PCT Filed: Aug. 10, 2007
(86) PCT No.: PCT/GB07/03042

§ 371 Date: Jul. 30, 2010

(30) Foreign Application Priority Data
Aug. 11, 2006 (GB) 0615957.8
Jul. 12, 2007 (GB) 0713593.2

(51) Int. Cl. A61K 31/555; C07F 15/00; A61K 33/24; A61K 31/66; A61K 31/704; A61K 31/7076; A61P 35/00

(52) U.S. Cl. 424/649; 546/4; 514/188; 514/110; 514/34; 514/48

(57) ABSTRACT

The present invention relates to osmium compounds of formula (I), their preparation and use in methods of treatment, particularly for cancer treatment.

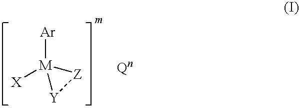

### FIELD OF INVENTION

The present invention relates to osmium compounds, their preparation and use in methods of treatment, particularly for cancer treatment.

### BACKGROUND OF THE INVENTION

Ruthenium compounds have been shown to have cytotoxic activity against human cancer cells. However, some ruthenium compounds have certain properties which may compromise their usefulness in cancer treatment.

Osmium complexes containing O,O-chelated ligands have been reported to be deactivated by loss of the chelate and formation of inert hydroxo-bridged dimers (Peacock A. F. A. et al, J. Am. Chem. Soc. 2006, 128, 1739-1748).

Accordingly, there is a need for alternative and/or improved compounds for cancer treatment.

The object of the present invention is to obviate and/or mitigate the problems seen with known ruthenium and osmium compounds in medical treatments, such as cancer treatment.

### SUMMARY OF THE INVENTION

According to a first aspect of the present invention there is provided a compound of formula (I):

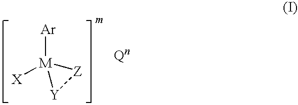

or a dinuclear or polynuclear form thereof, wherein,
M is an osmium(II) atom, or, in said dinuclear or polynuclear forms, at least one M is an osmium(II) atom,
Ar is an arene moiety,
X is halo, or a donor ligand,
Y————Z is a bidentate ligand, optionally linked to said arene moiety, wherein the dashed line represents a group of atoms linking Y and Z,
Y and Z are independently selected from O, N or S, with the proviso that Y and Z are not both O,
Q is an ion, and is either absent or present,
m and n are charges, independently either absent or selected from a positive or negative whole number,
or solvates or prodrugs thereof or physiologically active derivatives thereof, and
excluding the compounds having the following formula:

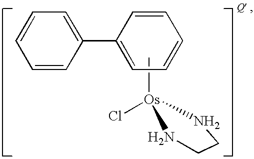

wherein Q′ is BF₄ (compound (II)) or BPh₄ (compound (III)).

The ligand Y———Z may be termed a Y,Z-chelated ligand. The proviso which requires that not both Y and Z are oxygen atoms, excludes compounds from the present invention containing O,O-chelated ligands. This is because the present applicant has found that such compounds readily form inert hydroxo-bridged dimers, and compounds which do not include such O,O-chelated ligands do not, or do not appreciably, form inert hydroxo-bridged dimers, and this provides an advantage over the O,O-chelated ligand complexes.

Furthermore, the present applicant has discovered that, although the structures of ruthenium compounds and the equivalent osmium compounds are almost identical, the reactivities of them are usually very different. Osmium complexes advantageously may undergo fewer side-reactions to allow more compound to reach a target site during therapy than the equivalent ruthenium compound resulting in improved usefulness as a pharmaceutical. The osmium complex [(η⁶-biphenyl)Os(en)Cl]⁺ (where en=ethylenediamine) has been found to hydrolyse 40 times more slowly than the equivalent ruthenium complex.

Additionally, it is found that the acidity of the coordinated water in [(η⁶-arene)M(YZ)(OH₂)]ⁿ⁺ (which is thought to be the active species) wherein M, Y and Z have the above meanings) is significantly lower for osmium compared to ruthenium (ca. 1.5 pKₐ units lower). The pH of blood and healthy cells is ca 7.4, however cancer cells have more acidic environments. Without wishing to be bound by theory, it is believed that the lower pKₐ values of water on osmium may allow the osmium complex to be present in an inactive form in the blood and healthy cells and activated upon entering cancer cells, whereas the ruthenium analogue may show more toxicity to healthy cells. The pKₐ may also affect the overall charge of the complex which is therefore likely to be different for osmium and ruthenium analogues in the body. The charge of a drug may be important in the overall pharmacological cell uptake and distribution. Both the different rates and charges associated with osmium complexes may result in different biological side reactions, pharmacological cell uptake and distribution.

The applicant has observed that osmium arene complexes are less coloured than the equivalent ruthenium analogues and potentially more light-stable, which may provide advantages in ease of formulation preparation and storage.

Also, arenes have been reported to be bound more tightly to osmium than to ruthenium, and therefore the loss of arene may be even less likely from osmium arene complexes, resulting in reduced side-effects.

Such properties as those described above may advantageously enable osmium complexes to be especially useful in a therapeutic setting.

The compounds according to formulae (II) and (III) are described by Peacock A. F. A. et al, J. Am. Chem. Soc. 2006, 128, 1739-1748, as inactive toward A2780 ovarian cancer cells, despite compound (III) being isostructural with an active Ru(II) analogue, i.e. of formula [(η⁶-biphenyl)Ru(en)Cl]BPh₄. The present inventors have now discovered that the reported results appear erroneous in that the compound of formula (II) does show cytotoxicity against A549 human lung and A2780 human ovarian cancer cell lines. The rationale for the erroneous results is that the test solution did not contain the desired compound, but instead contained a decomposition product(s) of the compound, apparently caused by the prevailing conditions during preparation and handling of the test solution.

According to a second aspect of the present invention there is provided a pharmaceutical composition comprising a compound according to the first aspect without excluding the compounds (II) and (III), together with a pharmaceutically acceptable carrier therefor.

The present invention, in a third aspect, provides a compound according to the first aspect without excluding the compounds (II) and (III) for use in medicine.

In a fourth aspect, the present invention provides the use of a compound according to the first aspect without excluding the compounds (II) and (III), for the preparation of a medicament for the treatment or prophylaxis of a disease involving cell proliferation, in particular cancer.

In a fifth aspect, the present invention provides a method of treatment or prophylaxis of a disease involving cell proliferation, in particular cancer, said method comprising administering a therapeutically or prophylactically useful amount of a compound according to the first aspect without excluding the compounds (II) and (III), to a subject in need thereof.

As an option, in the second to fifth aspects, the compounds (II) and (III) are excluded from the described uses/methods.

Preferably, when Y is O and Z is N, or vice versa, in the ligand, the N is a member of a saturated or unsaturated ring, i.e. the ring is an unsaturated or saturated heterocyclic ring.

A heterocyclic unsaturated ring is preferred, which is advantageously a heterocyclic aromatic ring, such as a pyridine ring.

The applicant has found that when the nitrogen atom in a N,O-ligand is the nitrogen in a nitrogen-containing heterocyclic ring, such as a pyridine ring, the osmium complex is stabilised against formation of inert hydroxo-bridged dimers.

The unsaturated or saturated ring may be substituted with one or more groups or fused or otherwise substituted to one or more further unsaturated or saturated rings, which may or may not be heterocyclic.

For example such N,O-ligand may have a structure (IV): (IV):

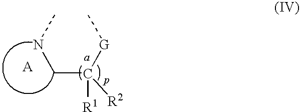

wherein, the ring A is a substituted or unsubstituted aromatic ring, optionally fused to one or more aromatic or saturated or unsaturated rings, and optionally includes one or more further heteroatoms in ring A or in the rings fused therewith;

G is O or NR³, wherein R³ is selected from the group consisting of hydrogen, branched or unbranched substituted or unsubstituted linear or cyclic alkyl, branched or unbranched substituted or unsubstituted linear or cyclic alkenyl, branched or unbranched substituted or unsubstituted linear or cyclic alkynyl or substituted or unsubstituted monocyclic or polycyclic aryl or heteroaryl;

R¹ and R² are independently selected at each occurrence from the group consisting of hydrogen, branched or unbranched substituted or unsubstituted linear or cyclic alkyl, branched or unbranched substituted or unsubstituted linear or cyclic alkenyl, branched or unbranched substituted or unsubstituted linear or cyclic alkynyl or substituted or unsubstituted monocyclic or polycyclic aryl or heteroaryl, carboxy, alkyloxycarbonyl hydroxyl, amino, nitro, alkyloxy, alkylthio, formyl, cyano, carbamoyl, halo (e.g. fluoro, chloro, bromo or iodo), —S(O)NR¹²R¹³ or —S(O) R¹⁴, or together, independently at each occurrence, form the group ═O or ═S, or independently may combine with ring A to form a ring fused with ring A, such fused ring being saturated or unsaturated, substituted or unsubstituted with any of the above-listed groups, and optionally includes one or more further heteroatoms;

p is a number from 1 to 6;

the bond labelled a is a single bond when both R¹ and R² on the carbon adjacent G are present or a double bond when one of R¹ and R² on the carbon adjacent G is absent; and the dashed lines represent the bonds to the metal (II) atom, e.g. Os(II).

More specifically, when G is O, the compound may have the structure (IVa):

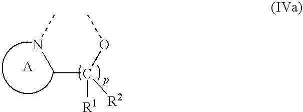

wherein, the ring A is a substituted or unsubstituted aromatic ring, which may be fused to one or more aromatic or saturated or unsaturated rings, and optionally includes one or more further heteroatoms in ring A or in the rings fused therewith.

The further heteroatoms are typically independently selected from nitrogen, oxygen and sulphur, preferably nitrogen.

R¹ and R² are independently selected at each occurrence from the group consisting of hydrogen, branched or unbranched substituted or unsubstituted linear or cyclic alkyl, branched or unbranched substituted or unsubstituted linear or cyclic alkenyl, branched or unbranched substituted or unsubstituted linear or cyclic alkynyl or substituted or unsubstituted monocyclic or polycyclic aryl or heteroaryl, carboxy, alkyloxycarbonyl hydroxyl, amino, nitro, alkyloxy, alkylthio, formyl, cyano, carbamoyl, halo (e.g. fluoro, chloro, bromo or iodo), —S(O)NR¹²R¹³ or —S(O)R¹⁴, or together, independently at each occurrence, form the group ═O or ═S, or independently may combine with ring A to form a ring fused with ring A, such fused ring being saturated or unsaturated, substituted or unsubstituted with any of the above-listed groups, and optionally includes one or more further heteroatoms;

p is a number from 1 to 6, such as 1 to 3, most preferably 1; and

the dashed lines represent the bonds to the metal (II) atom, e.g. Os(II).

Particularly preferred are complexes comprising a ligand in which p is 1, R¹ and R² together form a ═O group, and the ring A is a pyridine ring.

Examples of N,O-ligands include the following structures:

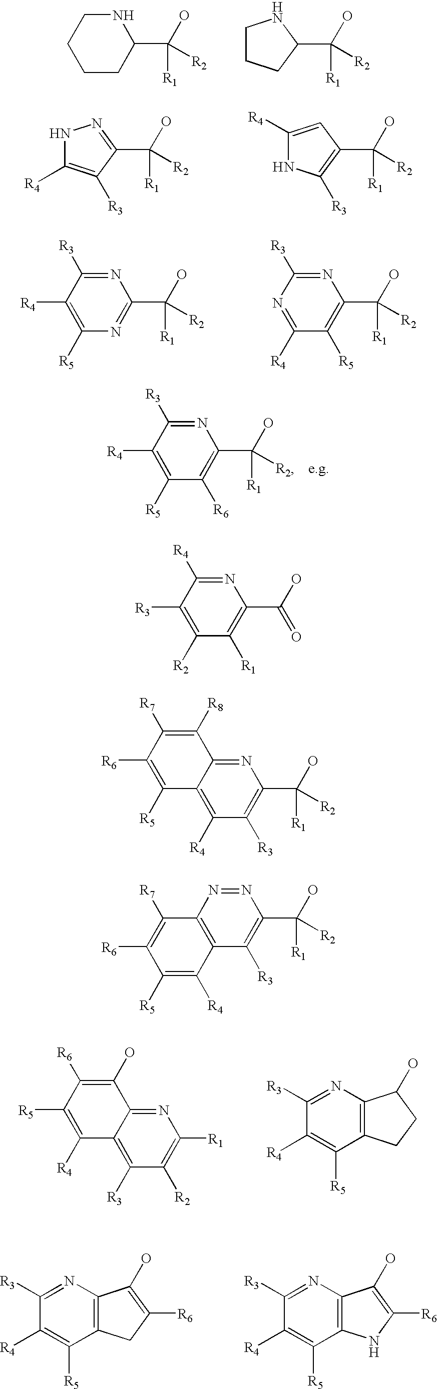

In the above structures, the R¹ and R² groups, have the same meaning as already indicated, and the remaining groups R³-R⁸ are independently at each occurrence selected from hydrogen, branched or unbranched substituted or unsubstituted linear or cyclic alkyl, branched or unbranched substituted or unsubstituted linear or cyclic alkenyl, branched or unbranched substituted or unsubstituted linear or cyclic alkynyl or substituted or unsubstituted monocyclic or polycyclic aryl or heteroaryl, carboxy, alkyloxycarbonyl hydroxyl, amino, nitro, alkyloxy, alkylthio, formyl, cyano, carbamoyl, halo (e.g. fluoro, chloro, bromo or iodo), —S(O)NR¹²R¹³ or —S(O)R¹⁴, wherein R¹³ and R¹⁴ are independently selected from H, branched or unbranched substituted or unsubstituted linear or cyclic alkyl, branched or unbranched substituted or unsubstituted linear or cyclic alkenyl, branched or unbranched substituted or unsubstituted linear or cyclic alkynyl, or substituted or unsubstituted monocyclic or polycyclic aryl or heteroaryl.

Alternatively, when the ligand is an N,N-ligand one or both of the nitrogen atoms are preferably part of a branched or unbranched, substituted or unsubstituted cyclic or straight chain aliphatic group, although aromatic rings are not excluded.

For example such N,N-ligand may have a structure (V):

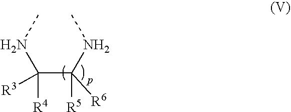

i.e. wherein the dashed line in formula (I) is —CR³R⁴—CR⁵R⁶—,

wherein R³, R⁴, R⁵ and R⁶ are independently selected at each occurrence from the group consisting of hydrogen, branched or unbranched substituted or unsubstituted linear or cyclic alkyl, branched or unbranched substituted or unsubstituted linear or cyclic alkenyl, branched or unbranched substituted or unsubstituted linear or cyclic alkynyl or substituted or unsubstituted monocyclic or polycyclic aryl or heteroaryl, carboxy, alkyloxycarbonyl hydroxyl, amino, nitro, alkyloxy, alkylthio, formyl, cyano, carbamoyl, halo (e.g. fluoro, chloro, bromo or iodo), —S(O)NR¹²R¹³ or —S(O)R¹⁴, or together, independently at each occurrence, form the group ═O or ═S or independently may combine with one or both of the donor nitrogen atoms to form a nitrogen-containing substituted or unsubstituted aliphatic or aromatic ring; and

p and the dashed lines have the same definitions as provided above.

Preferably, R³, R⁴, R⁵ and R⁶ are independently selected from hydrogen and alkyl, most preferably each is hydrogen.

Preferably p is 1.

When the ligand is an S,S-ligand, one or both of the sulphur atoms are bonded to a branched or unbranched, substituted or unsubstituted cyclic or straight chain aliphatic group, or an aromatic group.

For example, such ligand may have a structure (VI):

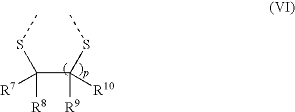

wherein,

R⁷, R⁸, R⁹ and R¹⁰ are independently selected at each occurrence from hydrogen, branched or unbranched substituted or unsubstituted linear or cyclic alkyl, branched or unbranched substituted or unsubstituted linear or cyclic alkenyl, branched or unbranched substituted or unsubstituted linear or cyclic alkynyl or substituted or unsubstituted monocyclic or polycyclic aryl or heteroaryl, carboxy, alkyloxycarbonyl hydroxyl, amino, nitro, alkyloxy, alkylthio, formyl, cyano, carbamoyl, halo (e.g. fluoro, chloro, bromo or iodo), —S(O)NR¹²R¹³ or —S(O)R¹⁴, or together, independently at each occurrence, form the group ═O or ═S or independently may combine with one or both of the donor sulphur atoms to form a sulphur-containing substituted or unsubstituted aliphatic ring; or R⁸ and R⁹ may combine to form an aromatic group, such as a phenylene ring, specifically an ortho-phenylene ring.

p and the dashed lines have the same definitions as provided above.

The cyclic aliphatic groups in the above definitions may contain one or more heteroatoms, e.g. nitrogen or oxygen.

Examples of N,O-ligand complexes include the following neutral structures:

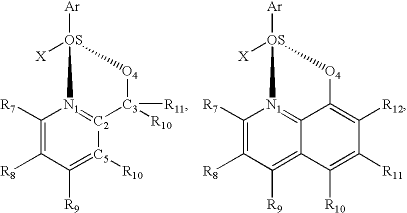

for example:

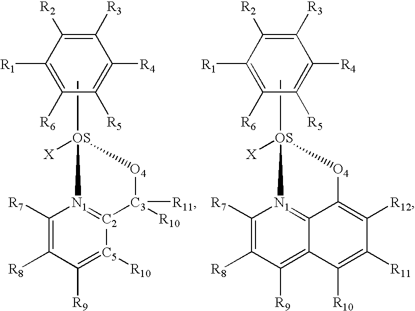

Particularly preferred N,O-ligands are picolinic acid or 8-hydroxyquinoline.

Examples of N,N-ligand complexes include the following cationic structures, together with a negative counter-ion Y:

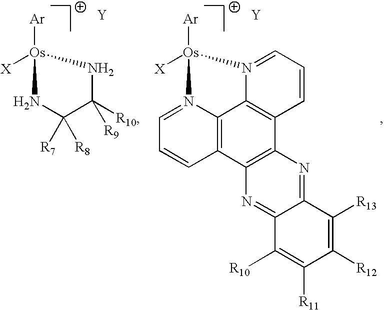

for example:

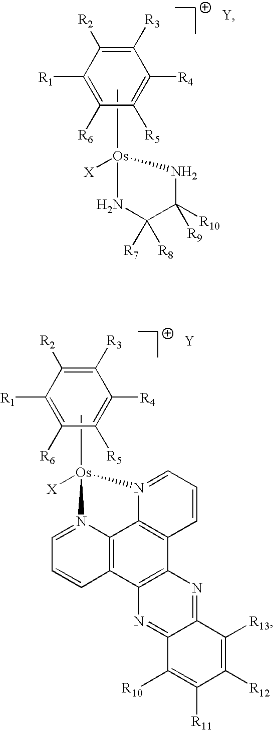

A particularly preferred N,N-ligand is ethylenediamine or substituted forms on one or both of the ethylene carbons and/or on one or both nitrogen atoms.

Examples of S,S-ligand complexes include the following anionic structures, together with a positive counter-ion Z:

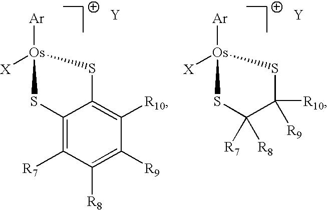

for example:

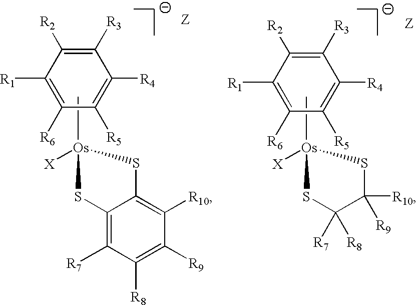

It will be appreciated that the ligand structure may be different when part of the metal complex as compared to the free ligand when not complexed. Thus, the ligand prior to being complexed may be termed a ligand precursor. For example, one or more hydrogens may be lost from a free ligand molecule to enable bonding to the metal atom to form the metal complex. As an example, picolinic acid, in the free state has the following structure:

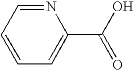

whereas when complexed to the metal, has the deprotonated structure:

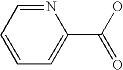

It will be appreciated that the complexed ligands may have a negative charge, which may be delocalised between the donor atoms, as will be understood by the skilled addressee according to known general principles.

The arene group (Ar), may be any arene, which may be substituted or unsubstituted. The above example structures show a benzene ring in which the R groups may be hydrogen or other substituents such as branched or unbranched substituted or unsubstituted linear or cyclic alkyl, branched or unbranched substituted or unsubstituted linear or cyclic alkenyl, branched or unbranched substituted or unsubstituted linear or cyclic alkynyl or substituted or unsubstituted monocyclic or polycyclic aryl or heteroaryl, carboxy, alkyloxycarbonyl hydroxyl, amino, nitro, alkyloxy, alkylthio, formyl, cyano, carbamoyl, halo (e.g. fluoro, chloro, bromo or iodo), —S(O)NR¹²R¹³ or —S(O)R¹⁴.

Examples of arene groups include benzene, naphthalene, anthracene, phenanthrene, fluoranthene, pyrene, triphenylene, fluorene, indene and the like. Other examples of suitable arene groups include biphenyl, cumene, styrene, mesitylene, cymene, toluene, xylene and the like. Yet further arene groups include partially de-hydro arene groups such as dihydronaphthalene (C₁₀H₁₀), tetradecahydroanthracene and 6,7-dihydro-5H-benzocyclo-heptane and the like.

The present invention also extends to compounds in which the arene group is tethered to the ligand moiety. The tether may be attached to the ligand at any position, including for example substituents or ring groups on the ligand.

For example, in an N,O-ligand having the structure (IV) above, the tether may be attached to a carbon atom of the ring A or to any of the R¹ or R² groups.

Tethers may also be used to provide dinuclear and polynuclear complexes in which at least one M is Os(II). All of the metal(II) atoms present may be Os(II) or alternatively, metals in addition to the at least one Os(II) (e.g. other group VIII transition metals in the period table) may be chosen, thus providing polyheteronuclear complexes.

Typically, in such compounds the other metal is Ru(II).

In such dinuclear and polynuclear complexes, the tethers may bridge between each of the complexes in any of a number of independent ways. For example, the tether may form a linkage between any one of the arene (Ar), chelating atom (Y or Z), chelating backbone (———) or directly from the metal (position X) in a first complex molecule to any one of those same positions in a second complex molecule, which is thereby joined or tethered to the first molecule.

Di- or polynuclear complexes containing both Os(II) and Ru(II) may be advantageous due to the differing properties and reactivities of the respective tethered Os(II) and Ru(II) complexes.

A tether may be represented by the group -{n}ₓ-, and examples of tethered dinuclear complexes include the following structures, in which M₁ and M₂ are both Os(II), or M₁ is Os(II) and M₂ is Ru(II) or vice versa:

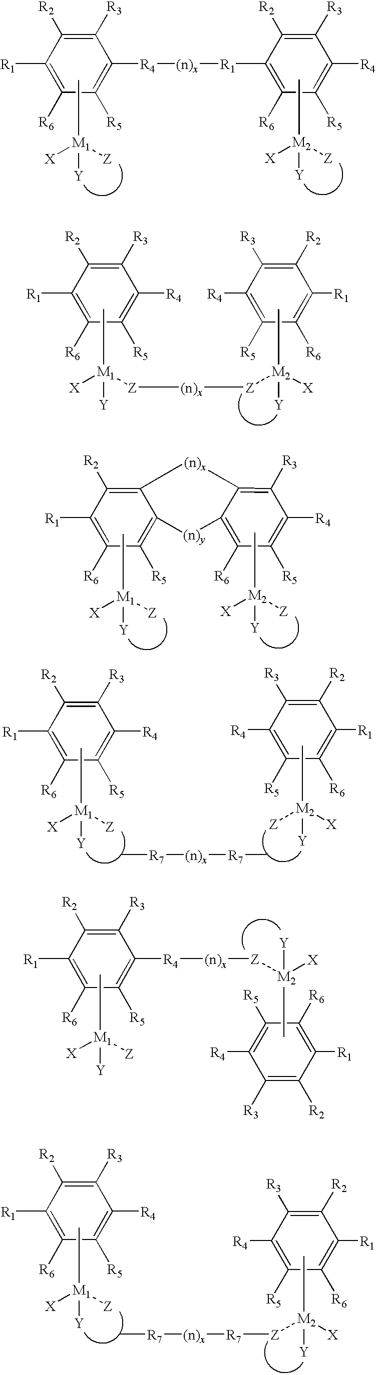

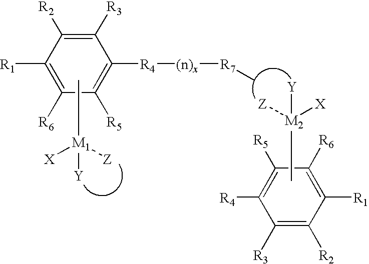

Internally tethered complexes may be represented by the following structures in which the tether is represented by a curved line:

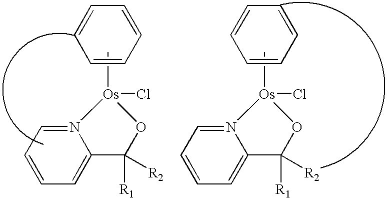

The tethers may be selected from any suitable group to provide a link between the respective desired groups of the complexes to be joined.

Typical tethers may be selected from alkylene, alkenylene, alkynylene, aromatic-containing groups, wherein the aromatic groups may optionally contain heteroatoms; and heteroatom-containing groups such as peptide and ether linkages.

The group X in the compounds according to formula (I) may be selected from the halogens i.e. fluoro, chloro, bromo or iodo. Alternatively, the group X may be selected from any suitable donor ligand, examples of which are ligands wherein the donor atom thereof is selected from the group consisting of nitrogen, oxygen, sulphur or phosphorous.

Typically, such ligand groups may be selected from pyridine (and derivatives thereof), water, hydroxo (i.e. OH⁻), azides or pseudohalogens and the like.

The group X may also be selected from nucleo-bases or derivatives thereof, e.g. a pyrimidine or purine, for example thymine, cytosine, adenine, guanine or uracil. Preferred examples include 9-ethylguanine and 9-ethyladenine.

The group X may be replaced by other groups when the compounds described herein are presented in a biological environment, for example, the species wherein X is water or hydroxo may be formed in a biological environment.

The ion, Q in compound according to formula (I), acts as a counter ion to the complex and balances the charges in the complex to generally provide a molecular species with overall charge of zero.

Negatively charged counter ions may be any suitable ion, for example selected from BF₄, BPh₄, PF₆, triflate and halides.

Positively charged counter ions may be any suitable ion, for example alkali metal cations such as Na⁺ and K⁺, or alkaline earth metal cations such as Mg²⁺ and Ca²⁺. Positive counter ions may also include organic cations, other metal complexes, protonated heterocyclic compounds and substituted or unsubstituted ammonium ions, i.e. NH₄⁺.

The counter ion may be chosen for certain purposes, for example, non-nucleophilic anions may be preferred, such as BPh₄ which tends to provide an insoluble complex thereby providing a useful advantage during a recovery stage of the compound preparation, e.g. for separation out of a solution or liquid medium. PF₆ may have a similar effect by providing a complex which is more soluble than a corresponding complex with BPh₄ counter ion, whilst remaining less soluble than a corresponding complex with chloride as the counter ion. These counter ions are not, however, necessarily excluded from the compound in its final usable form.

The counter ions may be chosen to provide a useful solubility for preparation of the complexes and the same counter ion may be retained or exchanged for another counter ion to provide a compound better suited for pharmaceutical/medical uses.

For example, triflate may be selected, or chloride, bromide or iodide to provide more easily soluble compounds.

Physiologically functional derivatives of compounds of the present invention are derivatives which can be converted in the body into the parent compound. Such physiologically functional derivatives may also be referred to as “pro-drugs” or “bioprecursors”. Physiologically functional derivatives of compounds of the present invention include in vivo hydrolysable esters. Additionally, the compounds of the present invention, may themselves, be considered as pro-drugs, which are converted into a physiologically active form in the body. Examples are the water (or aqua) complexes, i.e. where X is H₂O, which, without wishing to be bound by theory, are thought to be the active species in the biological environment.

It may be convenient or desirable to prepare, purify, and/or handle a corresponding solvate of the compounds described herein, which may be used in the any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

It will be appreciated that the compounds of the present invention may exist in various stereoisomeric forms and the compounds of the present invention as hereinbefore defined include all stereoisomeric forms and mixtures thereof, including enantiomers and racemic mixtures. The present invention includes within its scope the use of any such stereoisomeric form or mixture of stereoisomers, including the individual enantiomers of the compounds recited herein, as well as wholly or partially racemic mixtures of such enantiomers.

The compounds of the present invention may be prepared using reagents and techniques readily available in the art and as described hereinafter. Novel intermediate compounds in the synthetic route for preparation of the compounds of the present invention may be important molecules for general application for the preparation of the molecules of the present invention. Accordingly, the present invention extends to include those novel intermediate compounds.

The present invention also extends to the methods of preparing the compounds described herein. Generally, the method comprises providing a compound of formula ArOsX₂ in a first step and then reacting the compound with a ligand Y———Z in a second step to provide a compound according to formula (I).

The groups Ar, X, Y and Z have the same meaning as hereinbefore recited.

Preferably, in the preparation X in the starting material is halo, such as chloro.

During the preparation, a step may be included to exchange the counter ion of the complex for a different preferred counter ion.

Preferred preparation conditions comprise

i) providing and dissolving the compound ArOsX₂ with the ligand/ligand precursor in an alcoholic solvent, such as methanol, which may include an amount of water, optionally heating or refluxing the solution with or without stirring and for an amount of time as may be determined by the skilled addressee;

ii) introducing a suitable compound to the resultant mixture to add a preferred counter ion to the formed complex.

For example, a suitable compound for introducing the counter ion BF₄, is NH₄BF₄.

As indicated above, the present invention provides a treatment or prophylaxis of a disease, pathology or condition recited herein comprising administering a compound recited herein to a patient in need thereof.

Diseases involving abnormal proliferation of cells are treatable with the compounds recited herein. Examples of such diseases include cancers and hyperproliferation disorders.

Examples of cancers which may be treated by the active compounds include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, or skin, for example squamous cell carcinoma; a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma; a hematopoietic tumor of myeloid lineage, for example acute and chronic myelogenous leukemias, myelodysplastic syndrome, or promyelocytic leukemia; thyroid follicular cancer; a tumour of mesenchymal origin, for example fibrosarcoma or habdomyosarcoma; a tumor of the central or peripheral nervous system, for example astrocytoma, neuroblastoma, glioma or schwannoma; melanoma; seminoma; teratocarcinoma; osteosarcoma; xenoderoma pigmentoum; keratoctanthoma; thyroid follicular cancer; or Kaposi's sarcoma.

Examples of other therapeutic agents that may be administered together (whether concurrently or at different time intervals) with the compounds of the formula (I) include but are not limited to topoisomerase inhibitors, alkylating agents, antimetabolites, DNA binders and microtubule inhibitors (tubulin target agents), such as cisplatin, cyclophosphamide, doxorubicin, irinotecan, fludarabine, 5FU, taxanes, mitomycin C or radiotherapy. For the case of active compounds combined with other therapies the two or more treatments may be given in individually varying dose schedules and via different routes.

The combination of the agents listed above with a compound of the present invention would be at the discretion of the physician who would select dosages using his common general knowledge and dosing regimens known to a skilled practitioner.

Where the compound of the formula (1) is administered in combination therapy with one, two, three, four or more, preferably one or two, preferably one other therapeutic agents, the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer period apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

The patient is typically an animal, e.g a mammal, especially a human.

For use according to the present invention, the compounds or physiologically acceptable salt, ester or other physiologically functional derivative thereof described herein may be presented as a pharmaceutical formulation, comprising the compound or physiologically acceptable salt, ester or other physiologically functional derivative thereof, together with one or more pharmaceutically acceptable carriers therefore and optionally other therapeutic and/or prophylactic ingredients. The carrier(s) must be acceptable in the sense of being compatible with the other ingredients of the formulation and not deleterious to the recipient thereof.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an

active compound is formulated in an appropriate release-controlling matrix, or is coated with a suitable release-controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds.

Pharmaceutical formulations suitable for parenteral administration include sterile solutions or suspensions of an active compound in aqueous or oleaginous vehicles.

Advantageously, solutions may be prepared and stored in a ready to use condition, (e.g. without the need for further formulation such as dilution into a usable concentration), in light-excluding containers such as sealed bottles, ampoules, blister packages and the like. Such containers are preferably provided in a sterile condition.

Injectable preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

It should be understood that in addition to the aforementioned carrier ingredients the pharmaceutical formulations described above may include, an appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

Therapeutic formulations for veterinary use may conveniently be in either powder or liquid concentrate form. In accordance with standard veterinary formulation practice, conventional water soluble excipients, such as lactose or sucrose, may be incorporated in the powders to improve their physical properties. Thus particularly suitable powders of this invention comprise 50 to 100% w/w and preferably 60 to 80% w/w of the active ingredient(s) and 0 to 50% w/w and preferably 20 to 40% w/w of conventional veterinary excipients. These powders may either be added to animal feedstuffs, for example by way of an intermediate premix, or diluted in animal drinking water.

Liquid concentrates of this invention suitably contain the compound or a derivative or salt thereof and may optionally include a veterinarily acceptable water-miscible solvent, for example polyethylene glycol, propylene glycol, glycerol, glycerol formal or such a solvent mixed with up to 30% v/v of ethanol. The liquid concentrates may be administered to the drinking water of animals.

Embodiments of the present invention shall now be described with reference to the following non-limiting examples and the Tables, in which:

Example 1 describes the synthesis of compounds of the present invention;

Example 2 describes the x-ray crystallographic data obtained for compounds of the present invention;

Example 3 describes the properties of the compound of the present invention;

Example 4 describes the biological activity of the compounds of the present invention.

Example 5 describes Further work on the optimization of the biological activity of osmium(II) arene complexes where Y———Z (see formula (I)) represents picolinate derivatives.

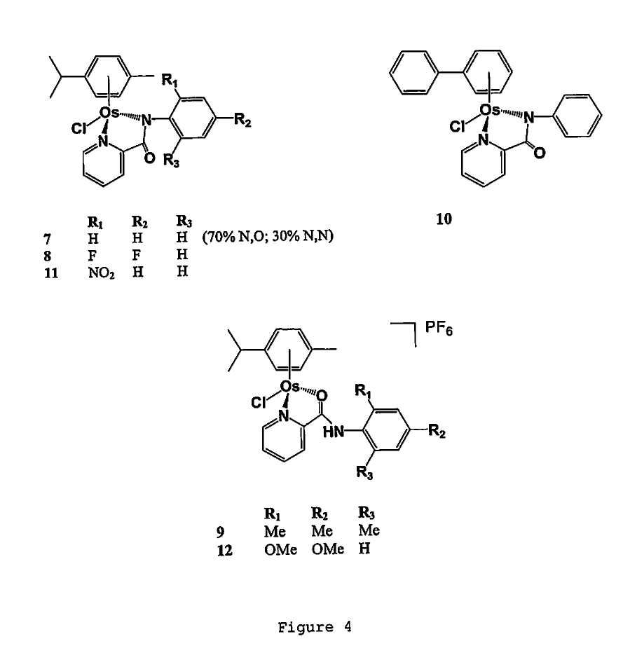

Example 6 describes studies on further compounds 8-12 shown in FIG. 4.

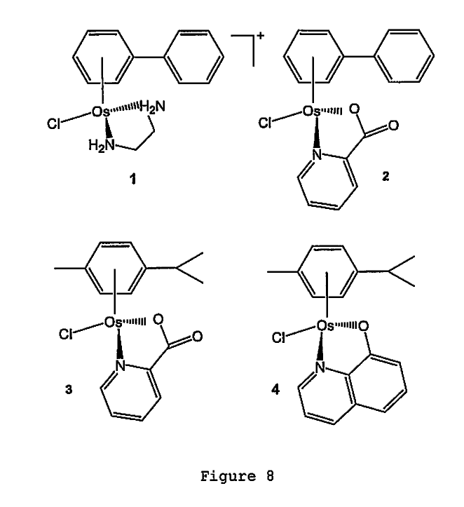

Example 7 describes Further cytotoxicity determinations evaluated for compounds 1-4 shown in FIG. 7.

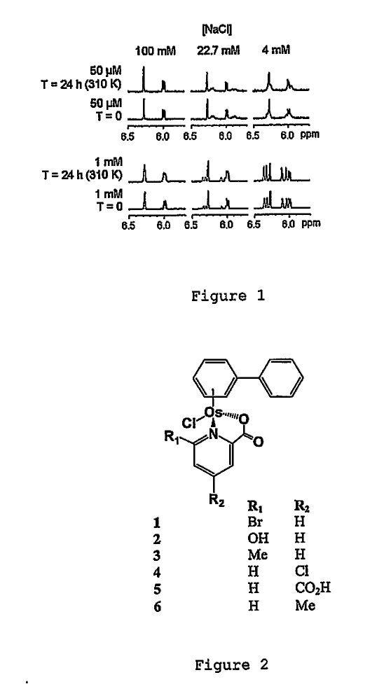

Examples 1 to 4 refer to FIG. 1 which shows the effects of chloride levels on the hydrolysis of AFAP42.

### EXAMPLE 1

### Synthesis

### [(η⁶-bip)Os(en)Cl]BF₄ (AFAP51)

A solution of [(η⁶-bip)OsCl₂]₂ (187 mg, 0.22 mmol) in 12 mL methanol, was refluxed for 80 min under argon, and ethylenediamine (32 μL, 0.48 mmol) was added and the reaction mixture heated for a further 40 min. The mixture was filtered through a 0.2 μm pore size filter while still hot, NH₄BF₄ (390 mg, ca 8 eq) was added, stirred, and the solvent removed in the rotary evaporator. Soxlett extraction with dichloromethane for 5.5 h. The solvent volume was reduced to ca 5 mL and stored at 253 K overnight. The yellow microcrystalline product was recovered by filtration, washed with dichloromethane (10 mL) and diethyl ether (10 mL) and air-dried. Yield: 129 mg (54%). Anal. Calcd for C₁₄ClH₁₈N₂OsBF₄ (526.794): C, 31.92; H, 3.44; N, 5.32%. Found: C, 32.05; H, 3.20; N, 5.07%. ¹H NMR (DMSO-d₆): δ=7.69 (d, 2H, J=7.2 Hz), 7.49 (t, 2H, J=7.6 Hz), 7.44 (t, 1H, J=7.3 Hz), 7.07 (b, 2H), 6.42 (d, 2H, J=5.7 Hz), 6.13 (t, 1H, J=5.0 Hz), 6.03 (t, 2H, J=5.3 Hz), 4.79 (b, 2H), 2.44 (m, 2H), 2.20 (m, 2H).

[(η⁶-bip)Os(en)Cl]PF₆ (AFAP05). Synthesis as above, replacing NH₄BF₄ with NH₄PF₆. Yield: 139 mg (67%) Anal. Calcd for C₁₄ClH₁₈N₂OsPF₆ (584.953): C, 28.75; H, 3.10; N, 4.79%. ¹H NMR (DMSO-d₆): δ=7.69 (d, 2H, J=7.2 Hz), 7.49 (t, 2H, J=7.4 Hz), 7.44 (t, 1H, J=7.3 Hz), 7.07 (b, 2H), 6.42 (d, 2H, J=5.5 Hz), 6.13 (t, 1H, J=5.1 Hz), 6.03 (t, 2H, J=5.4 Hz), 4.80 (b, 2H), 2.45 (m, 2H), 2.20 (m, 2H). Crystals suitable for X-ray diffraction were obtained by evaporation of a dichloromethane solution at ambient temperature.

[(η⁶-THA)Os(en)Cl]BF₄ (AFAP65). A solution of [(η⁶-THA)OsCl₂]₂ (27.1 mg, 0.03 mmol) in methanol (5 mL), was refluxed for 1.5 h under argon, and ethylenediamine (5 μL, 0.075 mmol) was added and the reaction mixture heated for a further 40 min. The mixture was filtered through a glass wool plug while still hot and a filtered solution of NH₄BF₄ (29 mg, ca 9 eq) in methanol (2 mL) was added, stirred, and the solvent removed in the rotary evaporator. Soxlett extraction with dichloromethane for 6.5 h. The solvent was allowed to slowly evaporate overnight (ca 0.5 mL). The microcrystalline product was recovered by filtration, washed with diethyl ether (10 mL) and air-dried. Yield: 6.4 mg (19%). Anal. Calcd for C₁₆ClH₂₂N₂OsBF₄ (554.847): C, 34.63; H, 4.00; N, 5.05%. Found: C, 34.33; H, 3.44; N, 4.87%. ¹H NMR (DMSO-d₆): δ=6.84 (b, 2H), 5.85 (dd, 2H, J=4.0 and 1.8 Hz), 5.78 (s, 2H), 5.73 (dd, 2H, J=3.9 and 1.9 Hz), 4.72 (b, 2H), 3.34 (m, 4H, J=175.1 and 16.4 Hz), 2.65 (s, 4H), 2.45 (m, 2H), 2.19 (m, 2H). Crystals suitable for X-ray diffraction were obtained by the evaporation of a dichloromethane solution.

[(η⁶-p-cym)Os(pico)Cl] (AFAP42). A solution of picolinic acid (18.6 mg, 0.15 mmol) and sodium methoxide (8.3 mg, 0.15 mmol) in MeOH (3 ml) was stirred at ambient temperature for 45 min and added to a solution of [(η⁶-p-cym)OsCl₂]₂ (50.8 mg, 0.06 mmol) in MeOH (5 ml) under argon. The resulting mixture was stirred at ambient temperature under argon for 20 h, the solvent removed on a rotary evaporator, the residue extracted with dichloromethane which was filtered through a cotton wool plug. The solvent reduced on a rotary evaporator to ca 3 mL and the product precipitated by the addition of diethyl ether. The yellow powder was recovered by filtration, washed with diethyl ether (10 mL) and air-dried. Yield: 55.8 mg (90%). Anal. Calcd for C₁₆ClH₁₈NO₂Os (482.003): C, 39.87; H, 3.76; N, 2.91%. Found: C, 39.87; H, 3.67; N, 2.80%. ¹H NMR (CDCl₃): δ=8.78 (d, 1H, J=5.7 Hz), 8.12 (d, 1H, J=7.6 Hz), 7.93 (td, 1H, J=7.6 Hz), 7.50 (td, 1H, J=6.4 Hz), 5.94 (d, 1H, J=5.7 Hz), 5.89 (d, 1H, J=5.7 Hz), 5.72 (d, 1H, J=5.7 Hz), 5.67 (d, 1H, J=5.7 Hz), 2.74 (sept, 1H, J=6.8 Hz), 2.35 (s, 3H), 1.22 (d, 3H, J=5.3 Hz), 1.21 (d, 3H, J=5.3 Hz). Crystals suitable for X-ray diffraction were obtained by evaporation of a chloroform/diethyl ether solution at ambient temperature in the dark.

[(η⁶-p-cym)Os(pico)(9EtG)]PF₆ (AFAP46). A solution of [(η⁶-p-cym)Os(pico)Cl] (32.6 mg, 0.07 mmol) and silver hexafluorophosphate (18.5 mg, 1.1 eq) in MeOH (3 ml) was stirred at ambient temperature for 5.5 h. The silver chloride precipitate was removed by filtration through a glass wool plug and added 9-ethylguanine (12.2 mg, 1.1 eq) was added to the resulting solution. The reaction mixture was stirred at ambient temperature under argon for ca 42 h. The resulting pale yellow precipitate was recovered by filtration, washed with methanol (3 mL) and diethyl ether (10 mL) and air-dried. Yield: 28.0 mg (54%). Anal. Calcd for C₂₃H₂₇N₆O₃OsPF₆ (770.693): C, 35.84; H, 3.53; N, 10.90%. Found: C, 35.74; H, 3.21; N, 11.23%. ESI MS: m/z: calcd for (C₂₃H₂₇N₆O₃Os)⁺: 627.2, found 627.2. ¹H NMR (MeOD-d₄): δ=9.64 (d, 1H, J=5.3 Hz), 8.12 (td, 1H, J=7.8 and 1.3 Hz), 7.95 (d, 1H, J=7.9 Hz), 7.85 (s, 1H), 7.74 (td, 1H, J=6.8 and 1.5 Hz), 6.50 (d, 1H, J=5.7 Hz), 6.24 (d, 1H, J=5.7 Hz), 6.12 (d, 1H, J=5.7 Hz), 6.04 (d, 1H, J=5.7 Hz), 4.13 (dq, 2H, J=7.3 and 2.6 Hz), 2.53 (sept, 1H, J=6.8 Hz), 2.04 (s, 3H), 1.39 (t, 3H, J=7.2 Hz), 1.17 (d, 3H, J=6.8 Hz), 1.03 (d, 3H, J=6.8 Hz). Crystals suitable for X-ray diffraction were obtained from diffusion of diethyl ether into a methanol solution at 277 K.

[(η⁶-p-cym)Os(pico)(9EtAd)]PF₆ (AFAP63). A solution of [(η⁶-p-cym)Os(pico)Cl] (24.9 mg, 0.05 mmol) and silver hexafluorophosphate (13.3 mg, 1.0 eq) in MeOH (3 ml) was stirred at ambient temperature for 2 h. The silver chloride precipitate was removed by filtration through a glass wool plug and added 9-ethyladenine (8.6 mg, 1.0 eq) was added to the resulting solution. The reaction mixture was stirred at ambient temperature under argon for ca 78 h. The yellow product was precipitated out by the addition of diethyl ether, recovered by filtration, washed with methanol (2 mL) and diethyl ether (10 mL) and air-dried. Yield: 13.3 mg (34%). Anal. Calcd for C₂₃H₂₇N₆O₂OsPF₆ (754.694): C, 36.60; H, 3.61; N, 11.14%. ¹H NMR (MeOD-d₄): δ=9.59 (d, 1H, J=5.3 Hz), 8.34 (s, 1H), 8.30 (s, 1H), 8.30 (overlapped t, 1H, ca 7 Hz), 8.06 (d, 1H, J=7.5 Hz), 8.01 (t, 1H, J=6.3 Hz), 6.64 (d, 1H, J=5.6 Hz), 6.52 (d, 1H, J=5.4 Hz), 6.16 (d, 1H, J=5.3 Hz), 6.11 (d, 1H, J=5.6 Hz), 4.63 (br s, 2H), 4.31 (q, 2H, J=7.0 Hz), 4.25 (q, 2H, J=7.0 Hz), 2.64 (sept, 1H, J=6.8 Hz), 1.85 (s, 3H), 1.41 (t, 3H, J=7.3 Hz), 1.17 (d, 3H, J=6.8 Hz), 1.01 (d, 3H, J=7.0 Hz). Crystals suitable for X-ray diffraction were obtained from diffusion of diethyl ether into a methanol solution at 277 K.

[(η⁶-p-cym)Os(quin)Cl] (AFAP62). A solution of 8-hydroxyquinoline (18.6 mg, 0.13 mmol) and sodium methoxide (6.9 mg, 0.13 mmol) in MeOH (2 ml) was added to a solution of [(η⁶-p-cym)OsCl₂]₂ (48.3 mg, 0.06 mmol) in MeOH (4 ml) and the resulting mixture stirred at ambient temperature for 5 h. The solvent was removed on a rotary evaporator, the residue extracted with acetone (ca 10 ml), solvent reduced on a rotary evaporator till a yellow precipitate starts to form, and stored at 253 K overnight. The yellow microcrystalline solid was recovered by filtration, washed with diethyl ether (5 mL) and air-dried. Yield: 43.5 mg (71%). Anal. Calcd for C₁₉ClH₂₀NOOs (504.051): C, 45.27; H, 4.00; N, 2.78%. Found: C, 45.35; H, 4.03; N, 2.68%. ¹H NMR (CDCl₃): δ=8.77 (d, 1H, J=4.9 Hz), 8.05 (d, 1H, J=8.3 Hz), 7.37 (t, 1H, J=7.9 Hz), 7.28 (dd, 1H, J=8.3 and 5.0 Hz), 7.03 (d, 1H, J=7.9 Hz), 6.84 (d, 1H, J=7.9 Hz), 5.93 (d, 1H, J=5.3 Hz), 5.82 (d, 1H, J=5.3 Hz), 5.70 (d, 1H, J=5.3 Hz), 5.63 (d, 1H, J=5.3 Hz), 2.62 (sept, 1H, J=6.8 Hz), 2.36 (s, 3H), 1.12 (dd, 6H, J=8.9 and 7.6 Hz).

[(η⁶-bip)Os(pico)Cl] (AFAP64). A solution of [(η⁶-bip)OsCl₂]₂ (51.3 mg, 0.06 mmol) in MeOH (5 ml) was refluxed under argon for 1 h before adding a solution of picolinic acid (16.8 mg, 0.14 mmol) and sodium methoxide (7.3 mg, 0.14 mmol) in MeOH (2 ml), which had been stirred at ambient temperature for 30 min. The resulting mixture was stirred at ambient temperature for 20 h, filtered through a cotton wool plug, the solvent removed on a rotary evaporator, the residue extracted with dichloromethane which was filtered through a cotton wool plug. The solvent removed again on a rotary evaporator, redissolved in methanol and the solvent volume reduced till a precipitate began to form. The vessel was stored at 278 K and the yellow powder was recovered by filtration, washed with diethyl ether (10 mL) and air-dried. Yield: 23.9 mg (39%). Anal. Calcd for C₁₈ClH₁₄NO₂Os (501.992): C, 43.07; H, 2.81; N, 2.79%. ¹H NMR (CDCl₃): δ=8.30 (d, 1H, J=5.4 Hz), 8.12 (d, 1H, J=7.9 Hz), 7.86 (t, 1H, J=7.7 Hz), 7.51 (m, 2H), 7.40 (m, 2H), 7.28 (7, 1H, J=6.6 Hz), 6.40 (d, 1H, J=5.3 Hz), 6.36 (d, 1H, J=4.9 Hz), 6.23 (t, 1H, J=5.0 Hz), 6.21 (t, 1H, J=5.0 Hz), 6.15 (t, 1H, J=5.0 Hz).

[(η⁶-p-cym)Os(O₂C₆H₃CH₃NCH₃)Cl] (AFAP57). A solution of 1,2-dimethyl-3-hydroxy-1,2-dimethyl-4(¹H)pyridone (17.6 mg, 0.13 mmol), sodium methoxide (6.8 mg, 0.13 mmol) and [(η⁶-p-cym)OsCl₂]₂ (47.7 mg, 0.060 mmol) in MeOH (4 mL) was stirred shielded from the light at ambient temperature for 26 h. The solvent removed on a rotary evaporator, the residue extracted with dichloromethane which was filtered through a glass wool plug. The solvent was again removed on a rotary evaporator and the product sonicated in acetone (10 mL). The yellow powder was recovered by filtration and washed with diethyl ether (10 mL) and acetone (10 mL) and air-dried. Yield: 34.2 mg (57%). Anal. Calcd for C₁₇ClH₂₂NO₂Os (498.05): C, 41.00; H, 4.45; N, 2.81%. ¹H NMR (CDCl₃): δ=6.97 (dd, 1H, J=6.8 and 0.9 Hz), 6.50 (dd, 1H, J=6.8 and 1.5 Hz), 5.99 (d, 1H, J=5.1 Hz), 5.91 (d, 1H, J=5.1 Hz), 5.77 (d, 1H, J=5.1 Hz), 5.71 (d, 1H, J=5.1 Hz), 2.72 (sept, 1H, J=7.0 Hz), 2.44 (d, 3H, J=1.2 Hz), 2.36 (d, 3H, J=1.2 Hz), 1.29 (d, 3H, J=6.6 Hz), 1.24 (d, 3H, J=6.6 Hz).

[(η⁶-p-cym)Os(S₂C₇H₆)Cl]Na (AFAP40). To a solution of [(η⁶-p-cym)OsCl₂]₂ (47.8 mg, 0.06 mmol) in MeOH (5 mL) was added sodium methoxide (6.7 mg, 0.124 mmol) and dithiotoluene (16.5 μL, 0.124 mmol). The solution instantly changed colour from clear yellow to a dark red. The solution was stirred at ambient temperature and shielded from the light for 3 h, before adding sodium chloride (70 mg, 10 eq) and stirring. The solvent was removed on a rotary evaporator and the residue extracted with diethyl ether, sonication assisted dissolution, filtered through a cotton wool plug and the vessel stored at 253 K overnight. The dark red powder recovered by filtration, washed with diethyl ether (5 mL) and air dried. Yield: 23.7 mg (37%). Anal. Calcd for C₁₇ClH₂₀NaOsS₂ (537.144): C, 38.01; H, 3.75%. ¹H NMR (CDCl₃): δ=8.04 (d, 1H, J=8.1 Hz), 7.97 (s, 1H), 6.92 (d, 1H, J=8.0 Hz), 6.05 (d, 2H, J=5.6 Hz), 6.01 (d, 2H, J=5.6 Hz), 2.63 (sep, 1H, J=6.8 Hz), 2.49 (s, 3H), 2.39 (s, 3H), 1.34 (d, 6H, J=6.8 Hz).

### EXAMPLE 2

### X-Ray Crystallographic Data:

TABLE 1
Crystallographic Data for [(η⁶-p-cym)Os(pic)Cl] AFAP42, [(η⁶-p-cym)Os(pic)
(9-EtG)]PF₆ AFAP46, [(η⁶-p-cym)Os(pic) (9-EtA)]PF₆ AFAP63 and [(η⁶-bip)Os(en)Cl]PF₆ AFAP05.

| | AFAP42 | AFAP46 | AFAP63•0.5Et₂O | AFAP05 |
|---|---|---|---|---|
| Formula | C₁₆H₁₈ClNO₂Os | C₂₃H₂₇F₆N₆O₃OsP | C₂₅H₂₃F₆N₆O_{2.5}OsP | C_{12.44}H₁₆Cl_{0.89}F_{5.33} |
| | | | | N_{1.78}Os_{0.89}P_{0.89} |
| Molecular weight | 481.98 | 770.67 | 791.74 | 1169.85 |
| Crystal description | Yellow block | Yellow block | Yellow block | Colourless block |
| Crystal size (mm) | 0.14 × 0.15 × 0.55 | 0.21 × 0.21 × 0.33 | 0.20 × 0.28 × 0.50 | 0.08 × 0.08 × 0.34 |
| λ (Å) | 0.71073 | 0.71073 | 0.71073 | 0.71073 |
| Temperature (K) | 150 | 150 | 150 | 150 |
| Crystal system | Monoclinic | Orthorhombic | Monoclinic, | Monoclinic |
| | | | twinned via 2(100) | |
| Space group | P 1 21/n 1 | Pna 21 | P 21/c | P1 21/c 1 |
| a (Å) | 10.1041(7) | 19.0626(3) | 12.2879(4) | 19.5853(8) |
| b (Å) | 15.0060(9) | 8.49080(10) | 15.9787(5) | 9.0300(4) |
| c (Å) | 10.2928(6) | 16.3786(3) | 15.1512(5) | 23.2542(8) |
| α (°) | 90 | 90 | 90 | 90 |
| β (°) | 99.521(3) | 90 | 98.317(2) | 112.608(2) |
| γ (°) | 90 | 90 | 90 | 90 |
| Volume (Å³) | 1539.12(17) | 2650.99(7) | 2943.57(16) | 3796.6(3) |
| Z | 4 | 4 | 4 | 9 |
| R | 0.042 | 0.043 | 0.0444 | 0.091 |
| R_{w} | 0.087 | 0.081 | 0.1118 | 0.097 |
| GOF | 0.760 | 0.757 | 1.114 | 0.681 |

TABLE 2
Selected Bond Lengths (Å) and Angles (°) for [(η⁶-p-cym)Os(pic)Cl]
AFAP42, [(η⁶-p-cym)Os(pic) (9-EtG)]PF₆ AFAP46, [(η⁶-p-cym)Os(pic)
(9-EtA)]PF₆ AFAP63 and [(η⁶-bip)Os(en)Cl]PF₆ AFAP05.

| Bond length/angle^{a} | AFAP42 | AFAP46 | AFAP63 | AFAP05 |
|---|---|---|---|---|
| Os—C(Arene) | 2.202(5) | 2.195(6) | 2.176(9) | 2.232(7) |
| | 2.170(5) | 2.195(6) | 2.176(9) | 2.208(7) |
| | 2.163(5) | 2.164(10) | 2.182(8) | 2.195(7) |
| | 2.180(5) | 2.229(6) | 2.184(8) | 2.176(8) |
| | 2.172(5) | 2.188(6) | 2.205(7) | 2.169(7) |
| | 2.174(5) | 2.180(6) | 2.206(8) | 2.194(7) |
| Os—X₁ | 2.090(4) | 2.101(5) | 2.094(7) | 2.136(6) |
| Os—X₂ | 2.080(3) | 2.097(7) | 2.081(7) | 2.152(6) |
| Os—X₃ | 2.4048(13) | 2.117(5) | 2.119(7) | 2.3918(19) |
| X₁—Os—X₂ | 77.33(15) | 76.0(2) | 76.9(2) | 78.7(2) |
| X₁—Os—X₃ | 83.18(11) | 84.1(2) | 81.8(3) | 83.47(17) |
| X₃—Os—X₂ | 83.85(11) | 80.6(2) | 85.2(2) | 83.65(18) |

^{a}X₁ = N and X₂ = N or O, X₃ = Cl or, for AFAP46 and AFAP63 X₃ = N(nucleobase)

TABLE 3
Crystallagraphic date for AFAP65.

| Empirical formula | C16 H22 B1 C11 F4 N2 Os1 |
|---|---|
| | [Os(Cl4H14) (C2H8N2)Cl] (BF4) |
| Formula weight | 554.82 |
| Wavelength | 0.71073 A |
| Temperature | 150 K |
| Crystal system | Monoclinic |
| Space group | P 1 21/n 1 |
| Unit cell dimensions | a = 12.722(3) A |
| | alpha = 90 deg. |
| | b = 7.9298(16) A |
| | beta = 105.28(3) deg. |
| | c = 18.142(4) A |
| | gamma = 90 deg. |
| Volume | 1765.6(7) A\hat{ }3 |
| Z | 4 |
| Density (calculated) | 2.087 Mg/m\hat{ }3 |
| Absorption coefficient | 7.414 mm\hat{ }−1 |
| F(000) | 1064 |
| B. DATA COLLECTION | |
| Crystal description | yellow plate |
| Crystal size | 0.44 × 0.15 × 0.09 mm |
| C. SOLUTION AND | |
| REFINEMENT. | |
| Solution | direct (SHELXS 86(Sheldrick, |
| | 1986)) |
| Refinement type | Full-matrix least-squares |
| | on F\hat{ }2 |
| Goodness-of-fit on F\hat{ }2 | 0.9498 |
| Conventional R [F > 4sigma(F)] | R1 = 0.0307 [3975 data] |
| Rw | 0.0907 |

TABLE 4
Bond lengths [A] and angles [deg] for AFAP65.

| | |
|---|---|
| Os(1)—Cl(1) | 2.4107(12) |
| Os(1)—N(2) | 2.140(4) |
| Os(1)—C(1) | 2.156(5) |
| Os(1)—N(1) | 2.127(4) |
| Os(1)—C(10) | 2.180(5) |
| Os(1)—C(11) | 2.224(4) |
| Os(1)—C(9) | 2.171(5) |
| Os(1)—C(14) | 2.218(5) |
| Os(1)—C(8) | 2.200(5) |
| Cl(1)—Os(1)—N(2) | 81.87(11) |
| Cl(1)—Os(1)—N(1) | 82.88(12) |
| N(2)—Os(1)—N(1) | 78.48(16) |

### EXAMPLE 3

### Solution Chemistry

### Acidity of Coordinated Water:

The mechanism established in the 70's for the mode of action of cisplatin, is thought to involve hydrolysis of the metal chloride bond. The active species is thought to be the aqua complex and not the deprotonated hydroxo species. Therefore, the complexes of the present invention for which the pKₐ of the coordinated water is ca 6.3 are present in the blood (pH ca. 7.4) as the less reactive/inert hydroxo species and on entering the cancer cells (pH ca. 6.3) are activated and can subsequently bind to DNA leading to cell death.

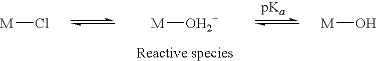

TABLE 5
pKₐ values for the water coordinated on
hydrolysis of the following complexes. The acidity
of water coordinated to osmium are ca 1.5 pKₐ units
lower than for the ruthenium corresponding
analogues.

| Aqua adduct of complex: | pKₐ |
|---|---|
| Osmium: | |
| AFAP51 | 6.34 |
| AFAP65 | 6.33 |
| AFAP52 | 6.29 |
| AFAP55 | 5.82 |
| AFAP41 | 5.81 |
| AFAP42 | 6.62 |
| AFAP64 | 6.30 |
| AFAP62 | 7.52 |
| AFAP43 | 7.17 |
| AFAP44 | 7.05 |
| AFAP60 | 7.08 |
| AFAP49 | 7.43 |
| AFAP23 | 7.60 |
| AFAP57 | 8.31 |
| AFAP29 | 7.63 |
| Ruthenium: | |
| RM175 | 7.71 |
| HCl1 | 8.01 |
| AH076 | |
| AH075 | |
| RF16 | |
| MM5 | |
| RF01 | 8.65 |
| RF02(a) | |
| RF11 | |
| MM45 | 9.23 |
| MMS6 | 9.4 |

The acidity of water coordinated to osmium appears to be highly tunable (pKₐ 5.8 to 8.3) by the choice of the chelating ligand. It is found that tuning the pKₐ to ca. 6.3-6.6, a value similar to that of the monoaqua adduct of cisplatin (6.41), results in cytotoxicity towards cancer cells. It is noted that the pKₐ of water coordinated to osmium is ca 1.5 pKₐ units more acidic than the corresponding ruthenium analogue. The aqua (OH₂) complex has one more overall positive charge than the hydroxo (OH) species, and without wishing to be bound by theory, the charge on osmium and ruthenium analogues in the body are likely to be different, which may affect the overall pharmacological cell uptake and distribution.

### Stability of AFAP42/62/64

Complexes containing O,O-chelated ligands are deactivated at micromolar concentrations by loss of the chelate and formation of inert hydroxo-bridged dimers. These dimers were also found to form in small amounts for the N,O-chelated glycine type complexes (AFAP43/44/60 and AFAP49) at micromolar concentrations. However, on introducing a pyridine ring into the chelate (AFAP42/62/62), 100% stability at 50 μM was achieved with no evidence of dimer formation even after incubation at 310 K (24 h).

Stability tests on AFAP42, 64, 62, 05, 51 and 65 in isotonic saline solutions were also performed. The solutions were found to be stable after 16 days of storage of the solutions in the dark.

### Effects of Chloride on AFAP42 in Solution

The effects of chloride levels on the distribution of AFAP42 (“prodrug”) and its aqua analogue (“active species”) were investigated. FIG. 1 shows the effect of 100 mM (chloride concentration in blood plasma), 22.7 mM (chloride concentration in cell cytoplasm), 4 mM (chloride concentration in the nucleus) at both 1 mM and the biologically relevant 50 μM AFAP42 initially and after incubation at 310 K (24 h). Results are summarised in table 6, showing how the hydrolysis of AFAP42 is suppressed in solutions with chloride concentrations similar to those found in blood plasma. At chloride concentrations typical of the cells cytoplasm, complex AFAP42 hydrolyses to some extent and at chloride concentrations typical of the nucleus (where the proposed target, DNA, can be found), the complex is present predominantly as the “active species” the aqua complex. FIG. 1 shows the effects of chloride levels on the hydrolysis of AFAP42.

TABLE 3
% of aqua complex formed at 100 mM, 22.7 mM
and 4 mM chloride concentrations, typically levels
found in blood plasma, cell cytoplasm and cell
nucleus respectively.

| | | 50 μM T = | | 1 mM T = |
|---|---|---|---|---|
| | 50 μM T = 0 | 24 h (310 K) | 1 mM T = 0 | 24 h (310 K) |
| 100 mM [Cl] | 0% Aqua | 0% Aqua | 12% Aqua | 12.9% Aqua |
| 22.7 mM [Cl] | 44.8% Aqua | 45.2% Aqua | 29.8% Aqua | 30.5% Aqua |
| 4 mM [Cl] | 73.5% Aqua | 72.1% Aqua | 56.1% Aqua | 65.5% Aqua |

### EXAMPLE 4

Experimental: Cytotoxicity Towards A2780 and A549 Human Cancer Cells. After plating, human ovarian A2780 cancer cells were treated with Os^{II} complexes on day 3, and human lung A549 cancer cells on day 2, at concentrations ranging from 2 μM to 50 μM. Solutions of the Os^{II} complexes were made up in 0.125% DMSO to assist dissolution. Cells were exposed to the complexes for 24 h, washed, supplied with fresh medium, allowed to grow for three doubling times (72 h), and then the protein content measured (proportional to cell survival) using the sulforhodamine B (SRB) assay.

The cytotoxicity data and complexes used in the A549 human lung and A2780 human ovarian cancer cell line experiments are shown in Table 7:

Cytotoxicity Data

TABLE 7
Cytotoxicity data complexes in the A549 human lung and
A2780 human ovarian cancer cell lines.

| | | IC₅₀/μM | IC₅₀/μM |
|---|---|---|---|
| Code | Structure | A549 | A2780 |
| Osmium N,N-chelates: | | | |
| AFAP05 | 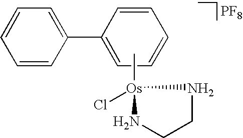 | 10 | 7 |
| AFAP51 | 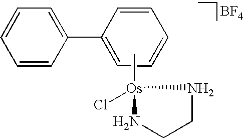 | 10 | 7.6 |
| AFAP65 | 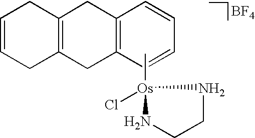 | 6.4 | 9.4 |
| AFAP52 | 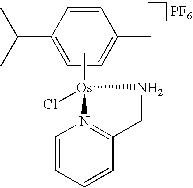 | >100 | >100 |
| AFAP55 | 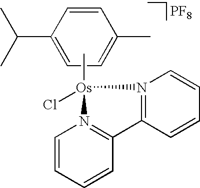 | >100 | >100 |
| AFAP41 | 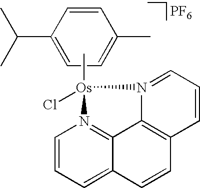 | >100 | >100 |
| Osmium N,O-chelates: | | | |
| AFAP42 | 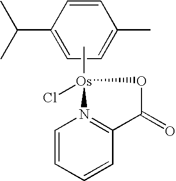 | 17 | 4.5 |
| AFAP64 | 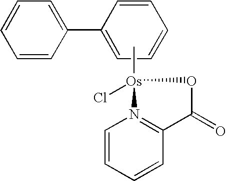 | 8 | 4.2 |
| AFAP62 | 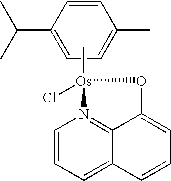 | 60 | 15.2 |
| AFAP43 | 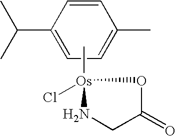 | >100 | >100 |
| AFAP44 | 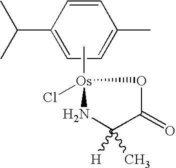 | >100 | >100 |
| AFAP60 | 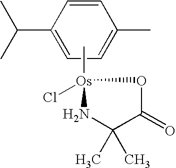 | >100 | >100 |
| AFAP49 | 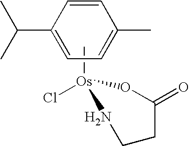 | >100 | >100 |
| Osmium O,O-chelates: | | | |
| AFAP23 | 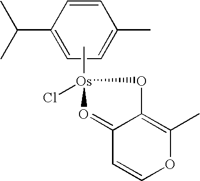 | >100 | >100 |
| AFAP57 | 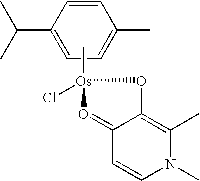 | >100 | 40 |
| AFAP29 | 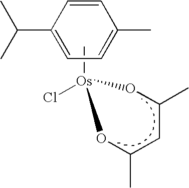 | >100 | >100 |
| Osmium S,S-chelates: | | | |
| AFAP40 | 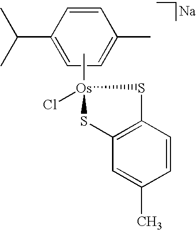 | Shows cytotoxicity. | |
| Ruthenium N,N-chelates: | | | |
| RM175 | 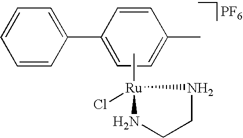 | 3.0 | 5 |
| HC11 | 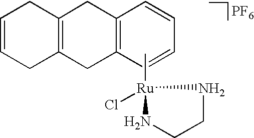 | 0.5 | 0.5 |
| AH076 | 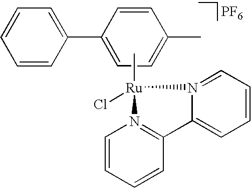 | | >100 |
| AH075 | 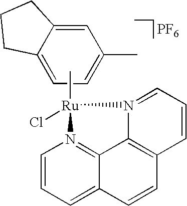 | | 52 |
| Ruthenium N,O-chelates: | | | |
| RF16 | 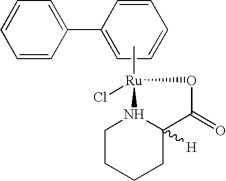 | | >100 |
| MM5 | 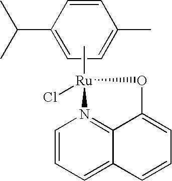 | | >100 |
| RF01 | 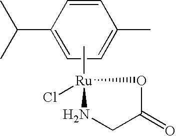 | | >100 |
| RF02(a) | 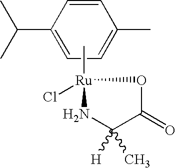 | | >100 |
| RF11 | 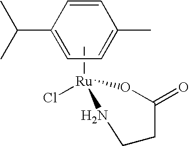 | | >100 |
| Ruthenium O,O-chelates: | | | |
| MM45 | 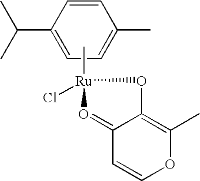 | >100 | >100 |
| MMS6 | 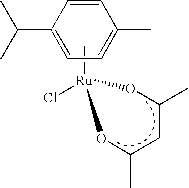 | >100 | 19 |

Osmium and ruthenium analogues do not show the same activity in vitro due to their different chemical behaviour in solution. The osmium compounds generally show greater cytotoxicity compared to the corresponding ruthenium complexes. AFAP62 shows poorer activity in the A549 cells but better activity (IC₅₀ 15 μM) in the A2780 cell line, whereas the ruthenium analogue (MM5) was less active (IC₅₀>100 μM) in the A2780 cell line. The ruthenium complex RF16, is not identical to AFAP64 but similar in structure and is less active, whereas AFAP64 shows greater activity in both cell lines.

AFAP65 is more active that the similar AFAP05 and AFAP51 and this may be due to the faster rates (eg hydrolysis) observed for this complex, ie more reactive, and the ability of the arene to intercalate into DNA as a secondary interaction/distortion. Complex AFAP64 is more active than AFAP42, and this is thought to be due again to a combination of intercalating arene as well as the now slower reactivity of the complex (eg hydrolysis). Maximum activity may be achieved for a complex with reactivity between AFAP65 and AFAP64 (as well as intercalating properties).

Both complexes containing the picolinic acid chelating ligand, AFAP42 and AFAP64, show the greatest activity in the A2780 cell line, whereas the ethylenediamine complex AFAP65 shows greatest activity in the A549 cancer cell line (with AFAP64 only slightly less active).

Solutions of AFAP40 (dark red solid, which has very poor aqueous solubility), were filtered to give a clear and colourless solution (presumably very low concentration of complex), yet showed activity when tested in vitro. This suggests that more water soluble complexes containing S,S-chelates and an overall negative charge may be highly active cytotoxics.

### New Activity Data for AFAP51:

Previously the complex AFAP51 was reported to be inactive against A2780 cells. Samples are prepared for testing by initially dissolving in 5% DMSO and subsequently diluting down. The applicant has found that solutions of AFAP51 in DMSO-d₆ can decompose when exposed to extreme conditions of heating, sonicating, exposure to air and extreme sunlight. It is possible that the initial result of inactivity may be the result of decomposition during sample preparation and before exposing the “drug solution” to the cells.

### EXAMPLE 5

Further work concerns the optimization of the biological activity of osmium(II) arene complexes where YZ (see formula (I))represents picolinate derivatives, which are cytotoxic towards cancer cells and were developed as potentially novel anticancer drugs. This has been approached by making systematic changes to their design by placing different substituents in the ortho- and para-position (R₁ and R₂) of the pyridine ring in the picolinate chelating ligand (FIG. 2), since these positions have been found to have significant electronic influence on the pyridine ring. The structure and numbering scheme of the osmium arene complexes studied in this work with picolinate derivatives as N,O-chelating ligands is shown in FIG. 2.

The methods and instrumentation for the studies in this example are the same as those described in Example 6 below.

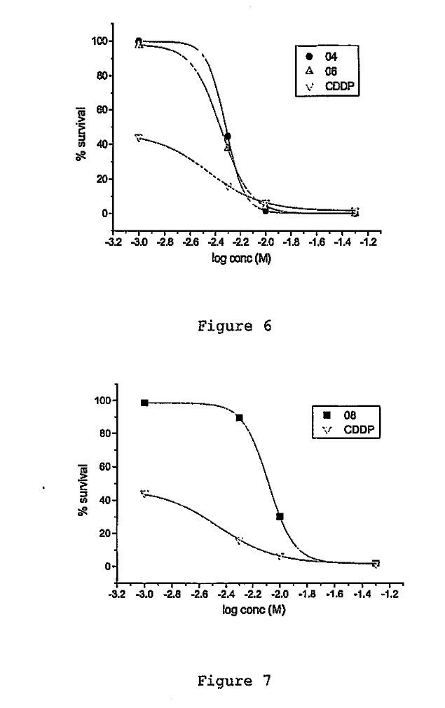

In this study, a substituent effect of the chloride, carboxyl and methyl groups present on the para-position of the pyridine ring (compounds 4, 5 and 6, respectively) with respect to the hydrolysis rates were observed, with a faster hydrolysis for the electron-donating methyl group and slower hydrolysis for the electron-withdrawing chloro and carboxyl groups compared to the unsubstituted parent compound (see Table 11). Of all 6 compounds tested, compounds 4 and 6 were found to be active in the human ovarian A2780 cancer cell line with IC₅₀ values of 4.8 and 4.4 μM respectively (See Table 8). FIG. 6 is a graph showing the IC₅₀ curves obtained. The cytotoxic inactivity in this particular study of compounds 1-3 may be caused by the presence of steric bulk caused by the ortho-substituents at the reactive site. This steric hindrance is further supported by their relatively slow hydrolysis rates and the reduced and weaker G and A binding observed for complex 3 compared to complex 6 (Table 14 and 15), which differ only by the ortho- and para-position of the methyl substituent on the picolinate ring.

This work shows that substituents on the picolinate backbone can have significant effects on the aqueous chemistry in osmium(II) compounds of the type [(η⁶-bip)Os(YZ)(Cl)] allowing a great scope for design for this class of compounds.

### Preparation of the Complexes

### Materials

1,4-Dihydrobiphenyl and the dimer, [(η⁶-bip)OsCl₂]₂, were prepared by previously reported procedures^{1,2} 9-Ethylguanine and 9-ethyladenine were purchased from Sigma-Aldrich. OsCl₃.nH₂O and 6-hydroxo picolinic acid (>97%) were purchased from Alfa Aesar. 2-cyano-4-methyl pyridine (98%) and thionyl chloride were obtained from Riedel de Haën and Fluka, respectively. 6-bromo picolinic acid (98%), 6-methyl picolinic acid (95%), 2,4 pyridinedicarboxylic acid monohydrate (98%) and all deuterated solvents were obtained from Aldrich. The ethanol and methanol were distilled over magnesium/iodine prior to use. Complexes 1-6 were synthesized from the dimeric precursor, [(η⁶-bip)OsCl₂]₂, using procedures similar to those reported previously for other half-sandwich Os(II) arene complexes.^{3,4}

### 4-Chloro picolinic acid

A suspension of picolinic acid (0.79 g, 6.35 mmol) and sodium bromide (1.30 g, 12.7 mmol) in 10 mL of thionyl chloride was heated under mild reflux for 20 h. The initially dark green mixture had changed to a dark red colour. Excess SOCl₂ was removed by rotary evaporation and the orange residue was taken up in about 15 mL of CH₂Cl₂ and was filtered through celite to get rid of any insoluble residues. The orange filtrate was cooled to −2° C. and 20 mL of H₂O (doubly distilled) was added dropwise while stirring vigorously, keeping the temperature between −2 and 2° C. The solution changed to a lighter orange colour and a white precipitate formed. The mixture was further stirred at r.t. for 20 h. The CH₂Cl₂ and H₂O were removed by rotary evaporation. The solid was recrystallised from a minimum amount of EtOH to give a yield of 0.28 g (28%).

¹H NMR (DMSO-d₆): δ=8.72 (1H, d, J=4.9 Hz), 8.09 (1H, d, J=1.6 Hz), 7.84 (1H, dd, J=4.9, 1.6 Hz)

### 4-Methyl picolinic acid

A solution of 2-cyano-4-methyl pyridine (0.15 g, 1.27 mmol) in about 10 mL of 6 M HCl was heated under reflux for 24 h. During this time, the initially light yellow solution changed to a clear solution. The solution was evaporated to dryness to leave a white solid. The solid was recrystallized from a minimal amount of distilled water to give a yield of 148 mg (85%). ¹H NMR (DMSO-d₆): δ=8.65 (1H, s), 8.06 (1H, s), 7.66 (1H, s) 2.05 (3H, s)

### [(η⁶-bip)Os(6-Br-pico)Cl] (1)

A solution of [(η⁶-bip)OsCl₂]₂ (51.9 mg, 0.06 mmol) in dry and degassed MeOH (10 mL) was heated under refluxed under argon for 1 hour before adding a solution of sodium methoxide (2.2 equiv, 7.2 mg) and 6-bromo picolinic acid (2.2 equiv, 27.2 mg) in 5 mL of dry and degassed MeOH. The resulting mixture was left refluxing mildly for 3 hours, filtered and solvent reduced on a rotary evaporator until precipitate began to form and was left standing at 278 K. The yellow powder was recovered by filtration and was air-dried to give a final yield of 45.6 mg (63%). Anal. Calcd for C₁₈H₁₃BrClNO₂Os (580.94): C, 37.22; H, 2.26; N, 2.41%. Found: C, 36.91; H, 2.12; N, 2.25%. ¹H NMR (CDCl₃): δ=8.09 (1H, d, J=7.0 Hz), 7.83 (1H, d, J=7.0 Hz), 7.70 (1H, t, 8 Hz), 7.52 (2H, d, J=7.9 Hz), 7.38 (3H, m), 6.71 (1H, d, J=5 Hz), 6.63 (1H, d, J=6 Hz), 6.46 (1H, t, J=5.29 Hz), 6.35 (1H, t, J=6.0 Hz), 6.32 (1H, t, J=5.29 Hz).

### [(η⁶-bip)Os(6-OH-pico)Cl] (2)

Synthesis as for 1 using [(η⁶-bip)OsCl₂]₂ (51.7 mg, 0.06 mmol), sodium methoxide (2.2 equiv, 6.71 mg) and 6-hydroxo picolinic acid (2.2 equiv, 19 mg). Yield: 37.3 mg (60%) ¹H NMR (DMSO-d₆): δ=13.12 (1H, br), 7.86 (1H, t, J=8 Hz), 7.65 (2H, d, J=7.2 Hz), 7.44 (3H, m), 7.33 (1H, d, J=7.0 Hz), 7.11 (1H, d, J=8.31 Hz), 6.74 (1H, d, J=5.29 Hz), 6.66 (1H, d, J=5.28 Hz), 6.42 (2H, m), 6.38 (1H, t, J=4.53 Hz).

### [(η⁶-bip)Os(6-Me-pico)Cl] (3)

Synthesis as for 1 using [(η⁶-bip)OsCl₂]₂ (52.2 mg, 0.06 mmol), sodium methoxide (2.2 equiv, 7.0 mg) and 6-methyl picolinic acid (2.2 equiv, 18 mg). Yield: 50 mg (77%) Anal. Calcd for C₁₉H₁₆ClNO₂Os (517.05): C, 44.22; H, 3.13; N, 2.71%. Found: C, 43.89; H, 2.65; N, 2.73%. ¹H NMR (CDCl₃): δ=7.95 (1H, d, J=7.93 Hz), 7.72 (1H, t, J=7.56), 7.43 (3H, m), 7.36 (3H, m), 6.51 (1H, d, J=5.29 Hz), 6.47 (1H, d, J=5.67 Hz), 6.30 (1H, t, J=5.29 Hz), 6.27 (1H, t, J=4.91 Hz), 6.22 (1H, t, J=5.29 Hz)

### [(η⁶-bip)Os(4-Cl-pico)Cl] (4)

Synthesis as for 1 using [(η⁶-bip)OsCl₂]₂ (51.5 mg, 0.06 mmol), sodium methoxide (2.2 equiv, 7.4 mg) and 4-chloro picolinic acid (2.2 equiv, 21.5 mg). Yield: 37.1 mg (54%) ¹H NMR (DMSO-d₆): δ=9.11 (1H, d, J=5.67 Hz), 7.93 (1H, d, J=1.89 Hz), 7.92 (1H, d*d, J=6.04*2.26 Hz), 7.65 (2H, m), 7.47 (3H, m), 6.76 (1H, d, J=5.29 Hz), 6.72 (1H, d, J=5.66), 6.46 (1H, t, J=5.29 Hz), 6.43 (1H, t, J=5.29 Hz), 6.40 (1H, t, J=4.91 Hz)

### [(η⁶-bip)Os(4-CO₂H-pico)Cl] (5)

Synthesis as for 1 using [(η⁶-bip)OsCl₂]₂ (53.6 mg, 0.06 mmol), sodium methoxide (2.2 equiv, 7.3 mg) and 2,4 pyridinedicarboxylic acid (2.2 equiv, 23.3 mg). Yield: 29.7 mg (41%) Anal. Calcd for C₁₉H₁₄ClNO₄Os (546.00): C, 41.80; H, 2.58; N, 2.57%. Found: C, 41.19; H, 2.12; N, 2.55%. ¹H NMR (DMSO-d₆): δ=9.31 (1H, m), 8.14 (1H, m), 8.01 (1H, m), 7.65 (2H, m), 7.47 (3H, m), 6.76 (1H, d, J=5.67 Hz), 6.72 (1H, d, J=6.04 Hz), 6.46 (1H, t, J=5.29 Hz), 6.44 (1H, t, J=5.29 Hz), 6.41 (1H, t, J=4.91 Hz).

### [(η⁶-bip)Os(4-Me-pico)Cl] (6)

Synthesis as for 1 using [(η⁶-bip)OsCl₂]₂ (53.1 mg, 0.06 mmol), sodium methoxide (2.2 equiv, 7.4 mg) and 4-methyl picolinic acid (2.2 equiv, 19.2 mg). Yield: 35.6 mg (57%) ¹H NMR (DMSO-d₆): δ=8.94 (1H, d, J=5.66 Hz), 7.74 (1H, br), 7.63 (2H, m), 7.52 (1H, d, J=4.53 Hz), 7.45 (3H, m), 6.73 (1H, d, J=5.29 Hz), 6.69 (1H, d, J=4.91 Hz), 6.40 (1H, m), 6.37 (2H, m).

### Cytotoxic Data

In this particular study, compounds 1-3 and 5 were found to be non-toxic in both human lung A549 and human ovarian A2780 cancer cell lines. Compounds 4 and 6, however were found to be toxic in the A2780 cancer cell line (Table 8) and FIG. 6. The steric bulk present around the metal centre caused by the ortho-substituents in the picolinate compounds 1-3 may account for their cytotoxic inactivity. The inactivity of compound 5 might be due to the ease of deprotonation of its para-substituent carboxyl group (pKₐ* 2.5), resulting in an overall negative charge which can result in less cellular uptake and once in the cell might be deactivated by binding to other biomolecules also present in the cell.

TABLE 8
In vitro growth inhibition of A2780 human ovarian
cancer cells and A549 human lung cancer cells with
compounds 1-6.

| | A2780 | A549 |
|---|---|---|
| Compound | IC₅₀ (μM) | IC₅₀ (μM) |
| 1 | >50 | >50 |
| 2 | >50 | >50 |
| 3 | >50 | >50 |
| 4 | 4.8 | 10-50* |
| 5 | >50 | >50 |
| 6 | 4.4 | 10-50* |

*Moderate activity was found for compounds 4 and 6 in the A549 cancer cell line.

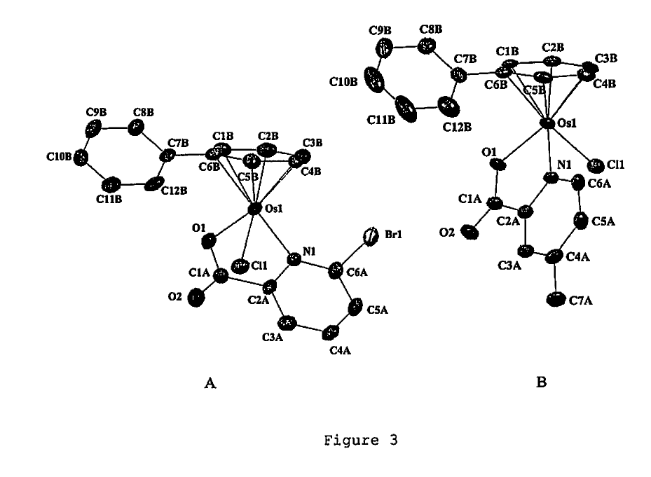

FIG. 3 shows X-ray crystal structure and atom numbering scheme for A) [(η⁶-bip)Os(6-Br-pico)Cl] (1), and B) [(η⁶-bip)Os(4-Me-pico)Cl] (6).

TABLE 9
Crystallographic data for [(η⁶-bip)Os(2-Br-
pico)Cl] (1) and [(η⁶-bip)Os(4-Me-pico)Cl] (6).

| | [(η⁶-bip)Os(2-Br- | [(η⁶-bip)Os(4-Me- |
|---|---|---|
| | pico)Cl] (1) | pico)Cl] (6). |
| Formula | C₁₉H₁₄BrCl₄NO₂Os | C₁₉H₁₆ClNO₂Os |
| Molecular | 700.22 | 558.44 |
| weight | | |
| Crystal | Colourless block | colourless lath |
| descirption | | |
| Size | 0.34 × 0.17 × 0.14 mm | 0.16 × 0.06 × 0.02 mm |
| λ (Å) | 0.71073 | 0.71073 |
| T/K | 150(2) | 120(2) |
| Crystal | Triclinic | Triclinic |
| system | | |
| Space group | P-1 | P-1 |
| a (Å) | 6.8555(8) | 8.065(5) |
| b (Å) | 11.0638(11) | 10.495(5) |
| c (Å) | 13.9209(19) A | 11.672(5) |
| α (°) | 99.783(7) | 81.806(5) |
| β (°) | 98.387(8) | 78.860(5) |
| γ (°) | 91.757(7) | 77.776(5) |
| Volume (Å³) | 1027.7(2) | 942.0(8) |
| Z | 2 | 2 |
| R | 0.0376 | 0.0399 |
| R_{w} | 0.0868 | 0.0837 |
| GOF | 0.997 | 1.120 |

TABLE 10
Selected bond lengths (Å) and angles (°) for
[(η⁶-bip)Os(2-Br-pico)Cl] (1) and [(η⁶-bip)Os(4-Me-pico)Cl] (6).

| Bond length/angle | Compound 1 | Compound 6 |
|---|---|---|
| Os(1)—C(1B) | 2.178(7) | 2.164(6) |
| Os(1)—C(2B) | 2.207(7) | 2.208(7) |
| Os(1)—C(3B) | 2.175(6) | 2.193(6) |
| Os(1)—C(4B) | 2.177(6) | 2.199(6) |
| Os(1)—C(5B) | 2.159(6) | 2.173(6) |
| Os(1)—C(6B) | 2.208(6) | 2.176(6) |
| Os(1)—O(1) | 2.077(4) | 2.082(4) |
| Os(1)—N(1) | 2.154(5) | 2.088(5) |
| Os(1)—Cl(1) | 2.4065(15) | 2.4095(19) |
| O(63A)—Os(1)—N(1A) | 77.35(18) | 77.10(18) |
| O(63A)—Os(1)—Cl(1) | 84.82(12) | 84.50(14) |
| N(1A)—Os(1)—Cl(1) | 81.74(13) | 83.30(15) |

### Hydrolysis

Since it is hypothesized that these types of complexes are activated in vivo by the equation of the chloro complex, the rates of hydrolysis of compounds 1-6 was determined. This was done in a 5% MeOD-d₄/95% D₂O mixture and the hydrolysis was monitored by ¹H NMR at 288 K or 298 K by the observation of new peaks over time due to aqua adduct formation. 5% MeOD was used to improve solubility and acidic conditions (D₂O of pH* 2) were used to prevent the deprotonation of the aqua complex as a secondary reaction.

All compounds except compound 2, [(η⁶-bip)Os(6-OH-pico)Cl], hydrolyse relatively fast with half-lives ranging from 0.98 to 4.4 h at 288 K. In addition, the equilibrium of hydrolysis lies to a great extent towards the aqua adducts for all compounds with even 100% hydrolysis observed for compounds 4 and 5, and in between 88% and 96% for 1, 3 and 6

TABLE 11
Hydrolysis data for compounds 1, 3-6 and that of
the parent compound at 288 K/298 K, determined by ¹H NMR.

| Compound | T/K | k/h⁻¹ | t_{1/2}/h |
|---|---|---|---|
| 1 | 298 | 0.345 ± 0.0002 | 2.01 ± 0.001 |
| 3 | 288 | 0.219 ± 0.005 | 3.17 ± 0.07 |
| | 298 | 0.676 ± 0.039 | 1.03 ± 0.04 |
| 4 | 288 | 0.288 ± 0.001 | 2.40 ± 0.08 |
| | 298 | 0.656 ± 0.032 | 1.06 ± 0.05 |
| 5 | 288 | 0.158 ± 0.011 | 4.42 ± 0.32 |
| | 298 | 0.384 ± 0.004 | 1.81 ± 0.02 |
| 6 | 288 | 0.710 ± 0.012 | 0.98 ± 0.02 |
| | 298 | 2.32 ± 0.08 | 0.30 ± 0.01 |

A substituent effect of the chloride, carboxyl and methyl groups present on the para-position of the pyridine ring (compounds 4, 5 and 6, respectively (Chart 1) with respect to the hydrolysis rates is observed, with a faster hydrolysis for the electron-donating methyl group and slower hydrolysis for the electron withdrawing chloro and carboxyl groups compared to the unsubstituted parent compound was observed. The electron-donating (or withdrawing properties) of the substituents introduces more (or less) electron density around the osmium centre compared to the parent compound which may promote chloride loss to a more (or lesser) extent

TABLE 12
Rate data for the aquation of active complex 6
at varying temperatures.

| | | | | Ea/ | ΔH^{‡}/ | ΔS^{‡}/ |
|---|---|---|---|---|---|---|
| Compound | T/K | k/h⁻¹ | t_{1/2}/h | kJ mol⁻¹ | kJ mol⁻¹ | J K⁻¹ mol⁻¹ |
| 6 | 278 | 0.17 | 4.06 | | | |
| | 285 | 0.44 | 1.57 | 90.58 | 87.7 | −55.56 |
| | 288 | 0.71 | 0.98 | | | |
| | 298 | 2.32 | 0.30 | | | |

The large negative activation entropy obtained for the hydrolysis of compound 6, suggests that the mechanism involves an associative pathway.

The effects of chloride concentrations typical of blood plasma (100 mM), cell cytoplasm (22.7 mM) and cell nucleus (4 mM) on the aqueous chemistry of 3 and 6 were investigated. ¹H NMR spectra of 3 or 6 (1 mM) were recorded within 10 min of sample preparation and after incubation at 310 K for 24 h.

TABLE 13
Percentage (%) of aqua adduct formation in a
solution of 1 mM 3 or 6 in D₂O at chloride levels typical
of blood plasma (100 mM), cell cytoplasm (22.7 mM) and
cell nucleus (4 mM).

| % aqua adduct | |
|---|---|
| t = 10 min | t = 26 h |

| [NaCl] | 3 | 6 | 3 | 6 |
|---|---|---|---|---|
| 100 mM | 35 | 10 | 27 | 20 |
| 22.7 mM | 60 | 53 | 49 | 53 |
| 4 mM | 50 | 82 | 70 | 81 |

### pH* Dependence

The changes in the ¹H NMR chemical shifts for the protons of the coordinated phenyl ring in compounds 3-6, present in an equilibrium of 3-6 and their aqua adduct as the major species, were followed with change in pH* over a range of 2-9. The chemical shift was plotted against pH*, and the resulting pH titration curves were fitted to the modified Henderson-Hasselbalch equation and gave rise to a pKₐ* value of 6.34 for 3, 6.30 for 4, 6.61 for 5 and 6.42 for 6. For compound 5 the pKₐ* value of the para-substituent carboxylate proton was also determined and was found to be 2.50.

Reactions with nucleobase models, 9-ethyl guanine (9EtG) and 9-ethyl adenine (9EtA)

Since DNA is believed to be the main target for metal anticancer drugs, nucleobase binding reactions of compounds 3-6, with nucleobase models, 9-ethyl guanine (9EtG) and 9-ethyl adenine (9EtA), were investigated.

Solutions of 3-6 (1 mM) (present as an equilibrium between 3-6 and their respective aqua adducts as the major species) with 1 mol equivalent of 9EtG (Table 14) or 9EtA (Table 15) in D₂O were prepared and ¹H NMR spectra were taken at different time intervals.

TABLE 14
Percentage (%) of 9EtG adduct formation with
compounds 3-6 at different time intervals.

| Compound | t = 10 | t = 24 h | t = 72 h |
|---|---|---|---|
| % 9EtG nucleobase adduct | | | |
| 3 | 0 | 30 | 30 |
| 4 | 0 | 59 | 76 |
| 5 | 0 | 100 | 100 |
| 6 | 0 | 80 | 85 |

TABLE 15
Percentage (%) of 9EtA adduct formation with
compounds 3-6 at different time intervals.

| Compound | t = 10 | t = 24 h | t = 72 h |
|---|---|---|---|
| % 9EtA nucleobase adduct | | | |
| 3 | 0 | 9 | 9 |
| 4 | 0 | 62 | 71 |
| 5 | 0 | 100 | 100 |
| 6 | 0 | 60 | 60 |

Active compounds 4 and 6 bind to a similar degree to nucleobase models 9EtG and 9EtA (Table 15). Compound 5 shows exceptionally high nucleobase affinity with 100% nucleobase adduct formation with both 9EtG and 9EtA after h indicating that its inactivity is not due to its nucleobase reactivity. For compound 3, the least amount of nucleobase binding was observed, with 30% binding to 9EtG and only 9% binding to 9EtA. This significant difference in nucleobase adduct formation of complex 3 compared to 4, 5 and 6 indicates a weaker binding of 3 to G and A bases possibly due to the steric bulk of the ortho-methyl group on the picolinate in compound 3 interfering with nucleobase binding.

### References for Example 5:

(1) Stahl, S.; Werner, H. Organometallics 1990, 9, 1876-1881.
(2) Peacock, A. F. A.; Habtemariam, A.; Fernandez, R.; Walland, V.; Fabbiani, F. P. A.; Parsons, S.; Aird, R. E.; Jodrell, D. I.; Sadler, P. J. Journal of the American Chemical Society 2006, 128, 1739-1748.
(3) Morris, R. E.; Aird, R. E.; Murdoch, P. D.; Chen, H. M.; Cummings, J.; Hughes, N. D.; Parsons, S.; Parkin, A.; Boyd, G.; Jodrell, D. I.; Sadler, P. J. Journal of Medicinal Chemistry 2001, 44, 3616-3621.
(4) Fernandez, R.; Melchart, M.; Habtemariam, A.; Parsons, S.; Sadler, P. L. Chemistry-a European Journal 2004, 10, 5173-5179.

### EXAMPLE 6

Further compounds 8-12 which have been studied are shown in FIG. 4.

### Experimental

### Materials

1,4-Dihydrobiphenyl and the dimers, [(η⁶-p-cym)OsCl₂]₂ and [(η⁶-bip)OsCl₂]₂, were prepared by previously reported procedures^{1,2} 9-Ethylguanine and 9-ethyladenine were purchased from Sigma-Aldrich. OsCl₃.nH₂O was purchased from Alfa Aesor. All deuterated solvents were obtained from Aldrich. The ethanol and methanol were distilled over magnesium/iodine prior to use. Complexes 8-12 were synthesized from the dimeric precursors, [(η⁶-p-cym)OsCl₂]₂ and [(η⁶-bip)OsCl₂]₂, using procedures similar to those reported previously for other half-sandwich Os(II) arene complexes.^{3,4}

### Methods and Instrumentation

Nuclear Magnetic Resonance (NMR) spectroscopy. ¹H NMR spectra were acquired in 5 mm NMR tubes at 298K (unless stated otherwise) on either a Bruker DMX 500 (¹H=500.13 MHz) or AVA 600 (¹H=599.81 MHz) spectrometer. ¹H NMR chemical shifts were internally referenced to (CHD₂)(CD₃)SO (2.50 ppm) for DMSO-d₆, CHCl₃ (7.26 ppm) for chloroform-d₁ and to 1,4-dioxane (3.75 ppm) for aqueous solutions. For NMR spectra taken in aqueous solutions, water suppression was carried out using Shaka or presaturation methods.⁵ All data processing was carried out using XWIN-NMR version 3.6 (Bruker U.K. Ltd).

Elemental analysis. Carbon, hydrogen and nitrogen (CHN) elemental analysis were carried out at The University of St. Andrews in the School of Chemistry on a Carlo Erba CHNS analyser.

X-ray crystallography. All diffraction data were collected by Dr. Simon Parsons and colleagues, University of Edinburgh, using a Bruker (Siemens) Smart Apex CCD diffractometer equipped with an Oxford Cryosystems low-temperature device operating at 150 K. Absorption corrections for all data sets were performed with the multi-scan procedure SADABS;⁶ structures were solved using either Patterson or direct methods (SHELXL⁷ or DIRDIF⁸); complexes were refined against F or F² using SHELXTL and H-atoms were placed in geometrically calculated positions. The modeling program Diamond 3.020 was used for production of graphics. The programs Mecury 1.4.1 and ORTEP 32 were used for analysis of data and production of graphics.

pH* measurements. pH* values (pH meter reading without correction for effects of D on glass electrode) of NMR samples in D₂O were measured at ca. 298 K directly in the NMR tube, before and after recording NMR spectra, using a Corning 240 pH meter equipped with a micro combination electrode calibrated with Aldrich buffer solutions of pH 4, 7 and 10.

Hydrolysis. The kinetics of hydrolysing complexes 8-12 was followed by ¹H NMR at different temperatures (298, 288 or 310 K). For this, solutions of the complexes with a final concentration of 0.8 mM in 5% MeOD-d4/95% D2O (v/v) were prepared by dissolution of the complexes in MeOD-d₄ followed by rapid dilution using D₂O with a pH* of ca 2, so that the aqua ligand was not deprotonated. ¹H NMR spectra were taken after various intervals using the presaturation method. The rate of hydrolysis was determined by fitting plots of concentrations (determined by ¹H NMR peak integrals) versus time to a pseudo first-order equation using ORIGIN version 5.0 (Microcal Software Ltd.).

pKₐ* calculations. For determinations of pKₐ* values (pKₐ values for solutions in D₂O), the pH* values of the aqua complex of 8 in D₂O (formed in situ by dissolution of the parent chloro complexes) were varied from ca pH* 2 to 10 by the addition of dilute NaOD and HNO₃, and ¹H NMR spectra were recorded. The chemical shifts of the arene ring protons were plotted against pH*. The pH* titration curves were fitted to the Henderson-Hasselbalch equation, with the assumption that the observed chemical shifts are weighted averages according to the populations of the protonated and deprotonated species. These pKₐ* values can be converted to pKₐ values by use of the equation pKₐ=0.929pKₐ*+0.42 as suggested by Krezel and Bal,⁹ for comparison with related values in literature.

Cancer Cell Cytotoxicity. After plating, human ovarian A2780 cancer cells were treated with OsII complexes on day 3, and human lung A549 cancer cells on day 2, at concentrations ranging from 0.1 μM to 100 μM. Solutions of the OsII complexes were made up in 0.125% DMSO to assist dissolution. Cells were exposed to the complexes for 24 h, washed, supplied with fresh medium, allowed to grow for three doubling times (72 h), and then the protein content measured (proportional to cell survival) using the sulforhodamine B (SRB) assay.10

Interactions with Nucleobases. The reaction of compounds 8-12 with nucleobases typically involved addition of a solution containing 1 mol equiv of nucleobase in D₂O, to an equilibrium solution of compounds 8-12 in D₂O (>90% aqua). The pH* value of the sample was adjusted if necessary so as to remain close to 7.4 (physiological). ¹H NMR spectra of these solutions were recorded at 298 K after various time intervals.

### Synthesis

### [(η⁶-p-cym)Os(N-2,4-difluoride-Ph-picolinamide)Cl]PF₆ (8)

A suspension of [(η⁶-p-cym)OsCl₂]₂ (51 mg, 0.064 mmol) and N-2,4-fluoride-Ph-picolinamide (2 equiv, 32 mg) in dry and degassed EtOH (25 ml) was refluxed under argon for 2 hours, after which the solution had turned colour from brown to yellow. The solution was filtered while hot into a filtered solution of NH₄PF₆ (˜5 equiv, 110 mg) in 5 ml of EtOH. The filtrate was reduced in volume on a rotary evaporator until a precipitate began to form and was left standing at 278 K. The orange crystalline powder was recovered by filtration and was air-dried to give a final yield of 42.9 mg (56%). ¹H NMR (DMSO-d₆): δ=9.25 (1H, d, J=5.67 Hz), 8.14 (1H, t*t, J=7.56*1.13 Hz), 7.91 (1H, d, J=7.55 Hz), 7.68 (1H, t*d, J=6.80*1.51 Hz), 7.53 (1H, qw, J=6.8 Hz), 7.26 (1H, t*d, J=10.2*3.0 Hz), 7.07 (1H, t*d, J=8.69*2.64 Hz), 6.04 (1H, d, J=4.91 Hz), 5.67 (1H, d, J=5.29 Hz), 5.51 (1H, d, J=5.29 Hz), 5.10 (1H, m), 2.42 (1H, q, J=6.8 Hz), 2.19 (3H, s) 0.97 (6H, t, J=6.8 Hz).

### [(η⁶-p-cym)Os(N-2,4,6-trimethyl-Ph-picolinamide)Cl]PF₆ (9)

A suspension of [(η⁶-p-cym)OsCl₂]₂ (50 mg, 0.063 mmol) and N-2,4,6-trimethyl-Ph-picolinamide (2 equiv, 29.9 mg) in dry and degassed EtOH (25 ml) was refluxed under argon for 2 hours, after which the solution turned colour from brown to yellow. The solution was filtered while hot into a filtered solution of NH₄PF₆ (˜5 equiv, 110 mg) in 5 ml of EtOH. The filtrate was reduced in volume on a rotary evaporator until a precipitate began to form and was left standing at 278 K. The yellow oily product was redissolved in a minimum of DCM and the volume was reduced until precipitate began to form and the vessel was left to precipitate out at 278 K. The orange crystals were recovered by filtration and air-dried to give a final yield of 59 mg (78%). Anal. Calcd for C₂₅H₃₀ClF₆N₂OOsP (745.17): C, 40.30; H, 4.06; N, 3.76%. Found: C, 40.52; H, 3.78; N, 3.67%. ¹H NMR (DMSO-d₆): δ=11.97 (1H, br), 9.55 (1H, br), 8.79 (1H, br), 8.49 (1H, br), 8.05 (1H, br), 7.09 (1H, br), 7.06 (1H, br), 6.49 (1H, br), 6.44 (2H, br), 6.24 (1H, br), 6.13 (1H, br), 2.58 (1H, m), 2.09(3H, s), 1.10 (6H, m). ³¹P NMR (DMSO-d₆) δ=−143 ppm (septet, J=Hz)

### [(η⁶-bip)Os(N-Ph-picolinamide)Cl]PF₆ (10)

A suspension of [(η⁶-bip)OsCl₂]₂ (50 mg, 0.06 mmol) and N-Ph-picolinamide (2 equiv, 25 mg) in dry and degassed EtOH (25 ml) was refluxed under argon for 2 hours. The brown solution was filtered while hot into a filtered solution of NH₄PF₆ (˜5 equiv, 110 mg) in 5 ml of EtOH. This mixture was stirred for another 3 hours. The mixture was reduced in volume on a rotary evaporator until a precipitate began to form and was left standing at 278 K. The orange precipitate was recovered by filtration and air-dried to give a final yield of 44.4 mg (64%). ¹H NMR (DMSO-d₆): δ=9.01 (1H, d. J=5.66 Hz), 8.04 (1H, t, J=7.93 Hz), 7.85 (1H, d, J=7.56 Hz), 7.53 (1H, m), 7.35 (3H, m), 7.30 (1H, m), 7.18 (1H, m), 7.08 (1H, s), 7.06 (1H, m), 6.99 (1H, s), 6.27 (1H, d, 5.29), 6.20 (1H, d, J=5.29 Hz), 5.98 (1H, t, J=4.91 Hz), 5.90 (1H, t, J=5.66 Hz), 5.79 (1H, t, J=5.28 Hz), 3.43 (1H, m), 1.91 (3H, s), 1.05 (6H, t, J=6.8 Hz).

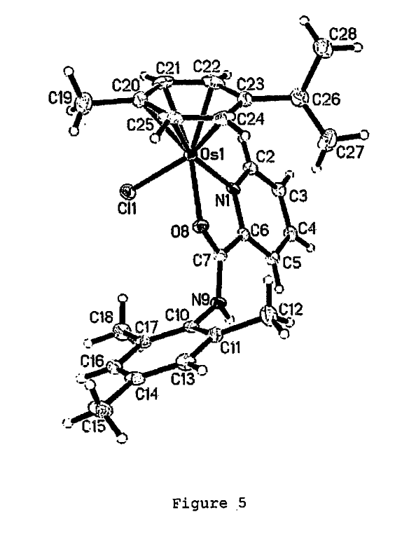

FIG. 5 depicts the X-ray crystal structure and atom numbering scheme for [(η⁶-p-cym)Os(N-2,4,6-trimethyl-Ph-picolinamide)Cl]PF₆ (9)

TABLE 16
Crystallographic data for [(η⁶-p-cym)Os(N-2,4,6-
trimethyl-Ph-picolinamide)Cl]PF₆ (9)

| | [(η⁶-p-cym)Os(N-2,4,6- |
|---|---|
| | trimethyl-Ph- |
| | picolinamide)Cl]PF₆ (9) |
| Formula | C₂₆H₃₂Cl₃F₆N₂O₁Os₁P₁ |
| Molecular | 830.07 |
| weight | |
| Crystal | Yellow block |
| descirption | |
| Size | 0.44 × 0.28 × 0.26 mm |
| λ (Å) | 0.71073 |
| T/K | 150(2) |
| Crystal | Monoclinic |
| system | |
| Space group | C c |
| a (Å) | 18.6715(4) |
| b (Å) | 10.7009(2) |
| c (Å) | 15.8714(3) |
| α (°) | 90 |
| β (°) | 105.3730(10) |
| γ (°) | 90 |
| Volume (Å³) | 3057.67(11) |
| Z | 4 |
| R | 0.0211 |
| R_{w} | 0.0540 |
| GOF | 0.6380 |

TABLE 17
Selected bond lengths (Å) and angles (°) for
[(η⁶-p-cym)Os(N-2,4,6-trimethyl-Ph-picolinamide)Cl]PF₆ (9)

| Bond length/angle | Compound 9 |
|---|---|
| Os(1)—C(20) | 2.216(4) |
| Os(1)—C(21) | 2.161(4) |
| Os(1)—C(22) | 2.158(4) |
| Os(1)—C(23) | 2.203(4) |
| Os(1)—C(24) | 2.170(4) |
| Os(1)—C(25) | 2.196(4) |
| Os(1)—O(8) | 2.117(2) |
| Os(1)—N(1) | 2.101(3) |
| Os(1)—Cl(1) | 2.3878(10) |
| O(8)—Os(1)—N(1) | 75.77(10) |
| O(8)—Os(1)—Cl(1) | 82.36(8) |
| N(1)—Os(1)—Cl(1) | 84.43(9) |

The following effects are considered in the complexes:
electron withdrawing substituents on the phenyl ring push N,N-coordination (compounds 8 and 11)
electron donating substituents push N,O-coordination (compound 9)
Biphenyl arene pushes N,N-coordination (compound 10)
   pKₐ* Determination.

The changes in the ¹H NMR chemical shifts for the protons of the coordinated phenyl ring in compound 8, present in an equilibrium of 8 and its aqua adduct as the major species, were followed with change in pH* over a range of 2-9. The chemical shift was plotted against pH*, and the resulting pH titration curves were fitted to the modified Henderson-Hasselbalch equation and gave rise to a pKₐ* value of 7.33 for 8

### Hydrolysis

The rates of hydrolysis of compounds 8-11 were determined. This was done in a 5% MeOD-d₄/95% D₂O mixture and the hydrolysis was monitored by ¹H NMR at 288 K or 310 K by the observation of new peaks over time due to aqua adduct formation.

TABLE 18
Hydrolysis data for compound 8, 10 and 11 at 288 K
and compound 9 at 310 K measured by ¹H NMR.

| Compound | k/h⁻¹ | t_{1/2}/h |
|---|---|---|
| 8 | ≧5.9 | ≦0.12 |
| 9 | 0.16 ± 0.01 | 4.23 ± 0.26 |
| 10 | ≧5.9 | ≦0.12 |
| 11 | ≧5.9 | ≦0.12 |

N,N-coordinated compounds 8, 10 and 11 hydrolyse fast (within 10 min at 288 K). N,O-coordinated compound 9 hydrolyses very slowly (Table 18).

### Cancer Cell Cytotoxicity.

Compound 8 was found to be cytotoxic in the human ovarian A2780 cancer cell line and moderately cytotoxic in the human lung A549 cancer cell line (Table 19).

TABLE 19
IC₅₀ values of 8 and 9 and cisplatin (CDDP) in A2780.

| Compound | IC₅₀ (μM) |
|---|---|
| 8 | 8.2 |
| 9 | >50 |
| CDDP | ~0.5 |

FIG. 7 shows IC₅₀ determination in A2780 of compound 8 and CDDP. Compound 8 is moderately active in human lung A549 cancer cell line (IC₅₀ in between 10-50 μM)

Binding to model nucleobases 9-ethyl guanine (9EtG) and 9-ethyl adenine (9EtA)

TABLE 20
percentage (%) of nucleobase adduct formation
measured at different time intervals by ¹H NMR

| | | +1 eq. 9-EtG (% | +1 eq. 9-EtA (% |
|---|---|---|---|
| Compound | Time interval | adduct formation) | adduct formation) |
| 8 | t = 0.17 h | 56 | 0 |
| | t = 24 h | 56 | 0 |
| 9 | t = 0.17 h | 0 | 0 |
| | t = 24 h | 0 | 0 |
| 10 | t = 0.17 h | 73 | 0 |
| | t = 24 h | 73 | 0 |
| 11 | t = 0.17 h | 69 | 0 |
| | t = 24 h | 69 | 0 |

No nucleobase binding is observed for inactive compound 9. Nucleobase adduct formation is observed for compounds 8, 10 and 11 with the equilibrium reached within 10 minutes and displaying guanine specificity.

From these results, it is noted that:

N,N- or N,O-coordination in Os(II) arene complexes containing N-Ph-picolinamide derivatives as chelating ligands (FIG. 4) is dependent on the substituents on the phenyl ring and/or on the coordinated arene.

N,N-Coordinated compounds 8, 10 and 11 hydrolyse fast (within 10 min at 288 K) and compound 8 is active in the A2780 cell line, while N,O-coordinated compound 9 hydrolyses very slowly (i.e. half-life time of 4.23 h at 310 K) and is inactive in this particular study.

Furthermore, compounds 8, 10 and 11 react extensively and exclusively with 9EtG while inactive compound 9 reacts with neither 9EtG or 9EtA indicating that N,N-coordination is needed for the wanted aqueous reactivity and cytotoxicity.

### References for Example 6:

(1) Stahl, S.; Werner, H. Organometallics 1990, 9, 1876-1881.
(2) Peacock, A. F. A.; Habtemariam, A.; Fernandez, R.; Walland, V.; Fabbiani, F. P. A.; Parsons, S.; Aird, R. E.; Jodrell, D. I.; Sadler, P. J. Journal of the American Chemical Society 2006, 128, 1739-1748.
(3) Morris, R. E.; Aird, R. E.; Murdoch, P. D.; Chen, H. M.; Cummings, J.; Hughes, N. D.; Parsons, S.; Parkin, A.; Boyd, G.; Jodrell, D. I.; Sadler, P. J. Journal of Medicinal Chemistry 2001, 44, 3616-3621.
(4) Fernandez, R.; Melchart, M.; Habtemariam, A.; Parsons, S.; Sadler, P. L. Chemistry-a European Journal 2004, 10, 5173-5179.
(5) Hwang, T. L.; Shaka, A. J. Journal of Magnetic Resonance Series A 1995, 112, 275-279.
(6) Sheldrick, G. M. University of Göttingen, Germany, 2001-2004.
(7) Sheldrick, G. M. SHELXL-97. Program for the refinement of crystal structures. University of Göttingen, Federal Republic of Germany. 1997.
(8) Beurskens, P. T.; Beurskens, G.; Bosman, W. P.; de Gelder, R.; Garcia-Granda; S.; Gould, R. O.; Israel, R.; Smits, J. M. M. Crystallography Laboratory, University of Nijmegen, The Netherlands. 1996.
(9) Krezel, A.; Bal, W. Journal of Inorganic Biochemistry 2004, 98, 161-166.
(10) Skehan, P.; Storeng, R.; Scudiero, D.; Monks, A.; McMahon, J.; Vistica, D.; Warren, J. T.; Bokesch, H.; Kenney, S.; Boyd, M. R. Journal of the National Cancer Institute 1990, 82, 1107-1112.

### EXAMPLE 7

Further cytotoxicity determinations were evaluated for compounds 1-4 shown in FIG. 8.

The human ovarian tumor cell lines A2780 (parent cisplatin sensitive) and A2780cisR (with acquired cisplatin resistance) were cultured in RPMI 1640 medium (Gibco), supplemented with 10% FBS, 2 mM glutamine, 50 μg/ml gentamycin at 37° C. in an atmosphere of 95% of air and 5% CO₂. Cell death was evaluated by using a system based on the tetrazolium compound MTT [3-(4,5-dimethyl-2-thiazolyl)-2,5-diphenyl-2H-tetrazolium bromide] which is reduced by living cells to yield a soluble formazan product that can be detected colorimetrically (Ref 1). Cells were seeded in 96-well sterile plates at a density of 10⁴ cells/well in 100 μl of medium and were incubated 16 h. Osmium complexes were dissolved in DMSO, the stock solutions were freshly prepared before use. The final concentration of DMSO in cell culture medium did not exceed 0.25%. The compounds were added to final concentrations from 0 to 128 μM in a volume of 100 μl/well. Seventy-two hours later 10 μl of a freshly diluted MTT solution (2.5 mg/ml) was pipetted into each well and the plate was incubated at 37° C. in a humidified 5% CO₂ atmosphere. After 5 h the medium was removed and the formazan product was dissolved in 100 μl of DMSO. The cell viability was evaluated by measurement of the absorbance at 570 nm, using an Absorbance Reader SUNRICE TECAN SCHOELLER. IC₅₀ values (compound concentration that produces 50% of cell growth inhibition) were calculated from curves constructed by plotting cell survival (%) versus drug concentration (μM). All experiments were made in quadruplicate.

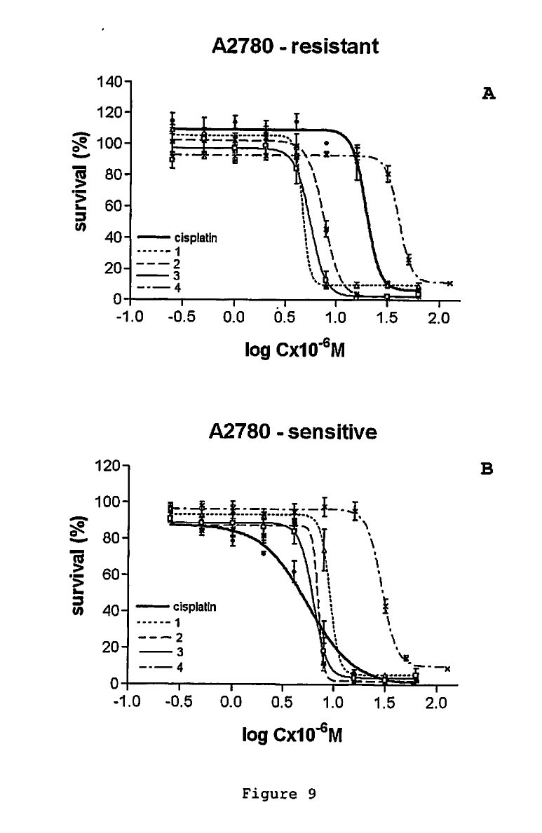

The results are shown graphically in FIG. 9 as dose response effects on the survival of A2780 (FIG. 9B) and A2780cisR (FIG. 9A) cancer cell lines. The cells were exposed to the osmium arene complexes and cisplatin for 72 hours in concentrations from 0 to 128 μM. Cell death was determined by MTT assay. The drug concentrations causing 50% inhibition (IC₅₀) were calculated. All experiments were performed in quadruplicate.

The results reported confirm the previously observed activity for these complexes, but here for the first time the activity in cisplatin resistant cells was also determined. That the osmium(II) arene complexes show very similar activity in both cell lines is highly significant and indicates a different detoxification mechanism for this class of complexes. Intriguingly complex 1, [(η⁶-bip)Os(en)Cl]⁺, shows even greater activity in the cisplatin resistant cell line, and such results indicate promising compounds with which to tackle the common problem of developed cisplatin resistance which can occur during chemotherapy treatment.

### Reference for Example 7:

1. Alley, M. C., Scudiero, D. A., Monks, A., Hursey, M. L., Czerwinski, M. J., Fine, D. L., Abbott, B. J., Mayo, J. G., Shoemaker, R. H. and Boyd, M. R. (1988) Feasibility of drug screening with panels of human tumor cell lines using a microculture tetrazolium assay. Cancer Res., 48, 589-601

The foregoing examples are provided as non-limiting illustrative embodiments of the present invention, which is not to be considered as limited thereby.

1-27. (canceled)

28. A compound of formula (I): 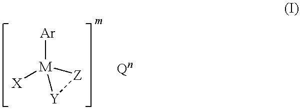
or a dinuclear or polynuclear form thereof, wherein,
M is an osmium (II) atom,
or, in said dinuclear or polynuclear forms, at least one M is an osmium (II) atom,
Ar is an arene moiety,
X is halo, or a donor ligand,
Y———Z is a bidentate ligand, optionally linked to said arene moiety, wherein the dashed line represents a group of atoms linking Y and Z,
Y and Z are independently selected from O, N or S, with the proviso that Y and Z are not both O,
Q is an ion, and is either absent or present,
m and n are charges, independently either absent or selected from a positive or negative whole number,
or solvates or prodrugs thereof or physiologically active derivatives thereof, and excluding the compounds having the following formula: 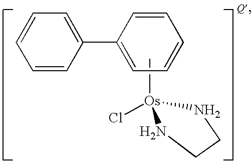
wherein Q′ is BF₄ (compound II) or BPh₄ (compound III).

29. The compound according to claim 28, wherein Y is N and Z is O or vice versa, and the N is a member of an aromatic ring.

30. The compound according to claim 29, wherein the Y———Z ligand has a structure (IV): 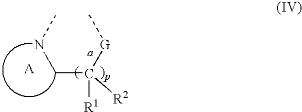
wherein, the ring A is a substituted or unsubstituted aromatic ring, optionally fused to one or more aromatic or saturated or unsaturated rings, and optionally includes one or more further heteroatoms in ring A or in the rings fused therewith;
G is O or NR³, wherein R³ is selected from the group consisting of hydrogen, branched or unbranched substituted or unsubstituted linear or cyclic alkyl, branched or unbranched substituted or unsubstituted linear or cyclic alkenyl, branched or unbranched substituted or unsubstituted linear or cyclic alkynyl or substituted or unsubstituted monocyclic or polycyclic aryl or heteroaryl;
R¹ and R² are independently selected at each occurrence from the group consisting of hydrogen, branched or unbranched substituted or unsubstituted linear or cyclic alkyl, branched or unbranched substituted or unsubstituted linear or cyclic alkenyl, branched or unbranched substituted or unsubstituted linear or cyclic alkynyl or substituted or unsubstituted monocyclic or polycyclic aryl or heteroaryl, carboxy, alkyloxycarbonyl hydroxyl, amino, nitro, alkyloxy, alkylthio, formyl, cyano, carbamoyl, halo (e.g. fluoro, chloro, bromo or iodo), —S(O)NR¹²R¹³ or —S(O)R¹⁴, or together, independently at each occurrence, form the group ═O or ═S, or independently may combine with ring A to form a ring fused with ring A, such fused ring being saturated or unsaturated, substituted or unsubstituted with any of the above-listed groups, and optionally includes one or more further heteroatoms;
p is a number from 1 to 6;
the bond labelled a is a single bond when both R¹ and R² on the carbon adjacent G are present or a double bond when one of R¹ and R² on the carbon adjacent G is absent; and the dashed lines represent the bonds to the metal (II) atom.

31. The compound according to claim 30, wherein p is 1.

32. The compound according to claim 30, wherein G is N and R³ is a substituted or unsubstituted phenyl ring.

33. The compound according to claim 32, wherein R¹ and R² together form the group ═O.

34. The compound according to claim 30, wherein G is O, the bond a is a double bond, one of R¹ or R² is absent and the other is amino substituted with a substituted or unsubstituted phenyl ring.

35. The compound according to claim 30, wherein the aromatic ring A is a substituted or unsubstituted pyridine ring or substituted or unsubstituted naphthalene ring.

36. The compound according to claim 28, wherein the ligand Y———Z is picolinate or 8-oxyquinoline.

37. The compound according to claim 28, wherein Y is N, Z is N, and the dashed line is —CR³R⁴—CR⁵R⁶—,
wherein R³, R⁴, R⁵ and R⁶ are independently selected at each occurrence from the group consisting of hydrogen, branched or unbranched substituted or unsubstituted linear or cyclic alkyl, branched or unbranched substituted or unsubstituted linear or cyclic alkenyl, branched or unbranched substituted or unsubstituted linear or cyclic alkynyl or substituted or unsubstituted monocyclic or polycyclic aryl or heteroaryl, carboxy, alkyloxycarbonyl hydroxyl, amino, nitro, alkyloxy, alkylthio, formyl, cyano, carbamoyl, halo (e.g. fluoro, chloro, bromo or iodo), —S(O)NR¹²R¹³ or —S(O)R¹⁴, or together, independently at each occurrence, form the group ═O or ═S or independently may combine with one or both of the donor nitrogen atoms to form a nitrogen-containing substituted or unsubstituted aliphatic or aromatic ring, wherein R¹³ and R¹⁴ are independently selected from H, branched or unbranched substituted or unsubstituted linear or cyclic alkyl, branched or unbranched substituted or unsubstituted linear or cyclic alkenyl, branched or unbranched substituted or unsubstituted linear or cyclic alkynyl, or substituted or unsubstituted monocyclic or polycyclic aryl or heteroaryl.

38. The compound according to claim 37, wherein R³, R⁴, R⁵ and R⁶ are each hydrogen.

39. The compound according to claim 28, wherein Y and Z are each S.

40. The compound according to claim 28, wherein Ar is selected from the group consisting of substituted or unsubstituted benzene, naphthalene, anthracene, phenanthrene, fluoranthene, pyrene, triphenylene, fluorene, indene, biphenyl, cumene, styrene, mesitylene, cymene, toluene, xylene, dihydronaphthalene (C₁₀H₁₀), tetradecahydroanthracene, 6,7-dihydro-5H-benzocyclo-heptane and the like.

41. The compound according to claim 35, wherein Ar is cymene or biphenyl.

42. The compound according to claim 1, wherein X is chloro.

43. The compound according to claim 28, wherein Q is a negatively charged ion selected from the group consisting of BF₄, BPh₄, PF₆, triflate and halides.

44. A method of preparing a compound according to formula (I): 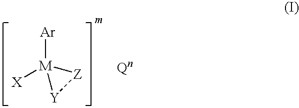
or a dinuclear or polynuclear form thereof, wherein,
M is an osmium (II) atom, or, in said dinuclear or polynuclear forms, at least one M is an osmium (II) atom,
Ar is an arene moiety,
X is halo, or a donor ligand,
Y———Z is a bidentate ligand, optionally linked to said arene moiety, wherein the dashed line represents a group of atoms linking Y and Z,
Y and Z are independently selected from O, N or S, with the proviso that Y and Z are not both O,
Q is an ion, and is either absent or present,
m and n are charges, independently either absent or selected from a positive or negative whole number, or solvates or prodrugs thereof or physiologically active derivatives thereof,
the method comprising providing a compound of formula ArOsX₂ in a first step and then reacting the compound with a ligand Y———Z in a second step to provide a compound according to formula (I).

45. A pharmaceutical composition comprising a compound according to claim 28, without excluding the compounds (II) and (III), together with a pharmaceutically acceptable carrier therefor.

46. A method of treatment or prophylaxis of a disease involving cell proliferation, said method comprising administering a therapeutically or prophylactically useful amount of a compound according to claim 1, without excluding the compounds (II) and (III), to a subject in need thereof.

47. The method according to claim 46, wherein the disease is cancer.

48. The method according to claim 47, wherein the cancer is selected from the group consisting of a carcinoma, a hematopoietic tumour of lymphoid lineage, a hematopoietic tumor of myeloid lineage, thyroid follicular cancer, a tumour of mesenchymal origin, a tumor of the central or peripheral nervous system, seminoma, teratocarcinoma, osteosarcoma, xenoderoma pigmentoum, keratoctanthoma, thyroid follicular cancer or Kaposi's sarcoma.

49. The method according to claim 46, wherein the compound according to formula (I) is administered together with one or more further therapeutic agents selected from the group consisting of topoisomerase inhibitors, alkylating agents, antimetabolites, DNA binders and microtubule inhibitors, or a therapeutic treatment.

50. The method according to claim 49, wherein the further therapeutic agent or the therapeutic treatment is selected from the group consisting of cisplatin, cyclophosphamide, doxorubicin, irinotecan, fludarabine, 5FU, taxanes, mitomycin C or radiotherapy.
